(19) United States
(12) Patent Application Publication
(10) Pub. No.: US 2014/0227233 A1
(43) Pub. Date: Aug. 14, 2014

(54) Treatment of Urinary Incontinence with Regenerative Glue

(71) Applicant: University Hospitals Cleveland Medical Center, Cleveland, OH (US)

(72) Inventor: Daneshgari Firouz, Bath, OH (US)

(73) Assignee: University Hospitals Cleveland Medical Center, Cleveland, OH (US)

(21) Appl. No.: 14/165,006

(22) Filed: Jan. 27, 2014

Related U.S. Application Data
(60) Provisional application No. 61/757,976, filed on Jan. 29, 2013.

(51) Int. Cl. A61L 24/00

(52) U.S. Cl. CPC A61L 24/0094; A61L 2430/10
USPC 424/93.7

(57) ABSTRACT

The invention relates to methods for the treatment of urinary incontinence (UI) and stress urinary incontinence (SUI) in both men and women by replacement of a damaged, absent or injured pubo-urethral ligament (PUL) via a simple injection of “Regenerative Glue.” For example, in the case of UI, the injection of “Regenerative Glue” at the PUL site will adhere the ventral surface of the urethra to the dorsal surface of the symphysis pubis (i.e. the space of Retzius), and further provide regenerative material for continuous replacement of deficient tissues and cells. “The Regenerative Glue” can be made from several commercially available materials including biocompatible glues, fibrin sealant or biocompatible gels; combined with Meschencymal stem cells (MSC) derived from bone marrow or adipose tissues.

### CROSS-REFERENCE TO RELATED APPLICATIONS

The present application claims the benefit of U.S. Provisional Patent Application No. 61/757,976, filed on Jan. 29, 2013, which is incorporated herein by reference.

### FIELD OF THE INVENTION

The invention relates to methods for the treatment of urinary incontinence (UI) and stress urinary incontinence (SUI) in both men and women by replacement of a damaged, absent or injured pubo-urethral ligament (PUL) via a simple injection of “Regenerative Glue.” For example, in the case of UI, the injection of “Regenerative Glue” at the PUL site will adhere the ventral surface of the urethra to the dorsal surface of the symphysis pubis (i.e. the space of Retzius), and further provide regenerative material for continuous replacement of deficient tissues and cells. For example, this “Regenerative Glue” may replace and potentially regenerate the PUL and other ligaments in the pelvis: a) an adhesive element similar in composition Ito connective tissue or the PUL and other ligaments; and b) a regenerative element that can interact with the surrounding environment (such as fibroblasts, fibromyoblasts, collagen, etc) to refurnish damaged or absent elements of the PUL and other ligaments. “The Regenerative Glue” can be made from several commercially available materials including biocompatible glues, fibrin sealant or biocompatible gels; combined with Meschencymal stem cells (MSC) derived from bone marrow or adipose tissues.

### BACKGROUND OF THE INVENTION

Female Pelvic floor disorders (FPFD) include a broad array of interrelated clinical conditions that includes urinary incontinence (UI), pelvic organ prolapse (POP), fecal incontinence (FI), sensory and emptying abnormalities of the lower urinary tract and defecatory dysfunction. Up to half of women experience one or more PFD for some period of their lives [1] and one in nine will undergo surgery for FPFD by age 80. UI is among the most prevalent conditions of the FPFD affecting approximately 40% of women in the United States and Western World [2]. Stress urinary incontinence (SUI) accounts for a large portion of these women. Stress urinary incontinence has also been found to be an issue with men, often after pelvic surgeries or trauma.

Surgical procedures including use of synthetic mesh has been increasingly used in treatment of POP and UI. For example, for treatment of SUI, mid-urethral slings (MUS), pioneered by the transvaginal tape sling procedure has been used recently. This procedure has enjoyed worldwide popularity with variation of route of entry and placement methodology to reduce the risks of complications. It is estimated that more than one million sling procedures have been performed worldwide, with approximately 250,000 surgical procedures being performed annually alone in the United States [3]. Despite the common use of mid-urethral slings, and other synthetic material in treatment of POP, use of these procedures are associated with a disturbing level of complications including mesh erosion, urinary retention, recurrence of SUI and urge urinary incontinence. Thus, numerous investigators and industrial partners are in active search for a less invasive and morbid treatment of SUI, and POP. Therefore, there is a continued need for improved treatments of pelvic floor disorders.

### SUMMARY OF THE INVENTION

The invention relates to methods for the treatment of urinary incontinence (UI) and stress urinary incontinence (SUI) in both men and women by replacement of a damaged, absent or injured pubo-urethral ligament (PUL) via a simple injection of “Regenerative Glue”. For example, in the case of UI, the injection of “Regenerative Glue” at the PUL site will adhere the ventral surface of the urethra to the dorsal surface of the symphysis pubis (i.e. the space of Retzius), and further provide regenerative material for continuous replacement of deficient tissues and cells. For example, this “Regenerative Glue” may replace and potentially regenerate the PUL and other ligaments in the pelvis: a) an adhesive element similar in composition to connective tissue or the PUL and other ligaments; and b) a regenerative element that can interact with the surrounding environment (such as fibroblasts, fibromyoblasts, collagen, etc) to refurnish damaged or absent elements of the PUL and other ligaments. “The Regenerative Glue” can be made from several commercially available materials including biocompatible glues, fibrin sealant or biocompatible gels; combined with Meschencymal stem cells (MSC) derived from bone marrow or adipose tissues.

In one embodiment, the invention relates to a method, comprising: a) providing: i) a subject exhibiting at least one symptom of urinary incontinence due to pubo-urethral ligament damage; ii) a composition comprising at least one fibrin glue and an amount of meschencymal stem cells (MSCs); b) contacting said composition with said pubo-urethral ligament damage; and c) reducing said at least one symptom of urinary incontinence. In one embodiment, said fibrin glue comprises elements selected from the group consisting of fibrinogen, collagen, collagen hydrogels, and tyramine substituted enzymatically cross-linkable collagen gels. In one embodiment, said MSCs are size-sieved stem cells (SSCs) with a diameter equal or greater than 3-μm. In one embodiment, said contacting is topical. In one embodiment, said reducing said at least one symptom enhances, at least partially, a functional recovery from urinary incontinence. In one embodiment, said reducing said at least one symptom improves urine retention. In one embodiment, said reducing said at least one symptom improves urine release. In one embodiment, said pubo-urethral ligament damage is caused by childbirth trauma. In one embodiment, said pubo-urethral ligament damage is caused by prostate surgery. In one embodiment, said meschencymal stem cells are derived from bone marrow or adipose tissue. In one embodiment, said urinary incontinence is stress urinary incontinence. In one embodiment, said fibrin glue composition is fresh.

In one embodiment, the invention relates to a composition comprising a fibrin glue and an amount of meschencymal stem cells (MSCs). In one embodiment, said fibrin glue comprises at least one element selected from the group consisting of fibrinogen, collagen, collagen hydrogels, and tyramine substituted enzymatically cross-linkable collagen gels. In one embodiment, said MSCs are size-sieved stem cells (SSCs) with a diameter equal or greater than 3-μm.

In one embodiment, the invention relates to a method for repairing pubo-urethral ligament damage and enhancing functional recovery from urinary incontinence in a subject, the method comprising providing a composition comprising a fibrin glue and an amount of meschencymal stem cells (MSCs). In one embodiment, said fibrin glue comprises elements including but not limited to fibrinogen, collagen, collagen hydrogels, and tyramine substituted enzymatically cross-linkable collagen gels, and wherein the MSCs are size-sieved stem cells (SSCs) with a diameter equal or greater than 3-μm. In one embodiment, the invention further comprises the step of applying the fibrin glue composition topically to the damaged pubo-urethral ligament to enhance at least partially the functional recovery from urinary incontinence, and result in at least one of improved urine retention and urine release of the subject. In one embodiment, said pubo-urethral ligament damage is caused by childbirth trauma. In one embodiment, said pubo-urethral ligament damage is caused by prostate surgery. In one embodiment, said meschencymal stem cells are derived from bone marrow or adipose tissue. In one embodiment, said urinary incontinence is stress urinary incontinence. In one embodiment, the invention further comprises preparing the fibrin glue composition freshly immediately before use.

In one embodiment, the invention relates to a composition for repairing pubo-urethral ligament damage and enhancing functional recovery from urinary incontinence in a subject, comprising a fibrin glue and an amount of meschencymal stem cells (MSCs). In one embodiment, said fibrin glue comprises elements including but not limited to fibrinogen, collagen, collagen hydrogels, and tyramine substituted enzymatically cross-linkable collagen gels, and wherein the MSCs are size-sieved stem cells (SSCs) with a diameter equal or greater than 3-μm.

### DEFINITIONS

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein, “collagen” refers to a group of naturally occurring proteins found in animals, especially in the flesh and connective tissues of mammals. It is the main component of connective tissue, and is the most abundant protein in mammals (Di Lullo, G. A. et al., 2002) [4], making up about 25% to 35% of the whole-body protein content. Collagen, in the form of elongated fibrils, is mostly found in fibrous tissues such as tendon, ligament and skin, and is abundant in cornea, cartilage, bone, blood vessels, the gut, and intervertebral disc.

As used herein, “collagen hydrogels” refers to tissue-like structure by contraction of collagen lattices by human fibroblasts of different proliferative potential in vitro as described in Bell, E. et al. (1979) [5]. Some examples of collagen hydrogels are described in Helary et al. (2010) [6].

As used herein, “fibrinogen” refers to a soluble plasma glycoprotein, synthesized by the liver that is converted by thrombin into fibrin during blood coagulation.

As used herein, “tyramine substituted enzymatically cross-linkable collagen gels” refers to specially modified collagen gels substituted with tyramine (4-hydroxyphenethylamine; para-tyramine, mydrial or uteramin). Some examples of tyramine substituted enzymatically cross-linkable collagen gels are described in Darr, A. and Calabro, A. (2009) [7].

As used herein, “childbirth trauma” refers to bodily injury as the result of childbirth.

As used herein, “prostate surgery” refers to surgery related to the prostate and may include prostate removal.

As used herein, “meschencymal stem cells” refers to multipotent stem cells that can differentiate into a variety of cell types [8], including: osteoblasts (bone cells) [9], chondrocytes (cartilage cells) [10], and adipocytes (fat cells).

As used herein, “bone marrow” refers to the flexible tissue found in the interior of bones.

As used herein, “adipose tissue” or “body fat” or “fat depot” or “fat” refers to loose connective tissue composed of adipocytes.

As used herein, “urinary incontinence” (abbreviated UI) refers to any involuntary leakage of urine. It can be a common and distressing problem, which may have a profound impact on quality of life. Urinary incontinence usually results from an underlying treatable medical condition but is under-reported to medical practitioners.

As used herein, “stress urinary incontinence” (abbreviated SUI), also known as effort incontinence, refers to a urinary incontinence condition due essentially to insufficient strength of the pelvic floor muscles.

It is the loss of small amounts of urine associated with coughing, laughing, sneezing, exercising or other movements that increase intra-abdominal pressure and thus increase pressure on the bladder. The urethra is supported by fascia of the pelvic floor. If this support is insufficient, the urethra can move downward at times of increased abdominal pressure, allowing urine to pass. Most lab results such as urine analysis, cystometry and postvoid residual volume are normal. Some sources distinguish between urethral hypermobility and intrinsic sphincter deficiency [11]. The latter is rarer, and requires different surgical approaches.

Stress incontinence is rare in men. The most common cause is as a post-surgical complication following a prostatectomy. In women, physical changes resulting from pregnancy, childbirth, and menopause often contribute to stress incontinence. Stress incontinence can worsen during the week before the menstrual period. At that time, lowered estrogen levels may lead to lower muscular pressure around the urethra, increasing chances of leakage. The incidence of stress incontinence increases following menopause, similarly because of lowered estrogen levels. In female high-level athletes, effort incontinence occurs in all sports involving abrupt repeated increases in intra-abdominal pressure that may exceed perineal floor resistance [12].

As used herein, “pubourethral ligament transaction” is abbreviated PULT.

As used herein, the term “tissue” is used throughout the specification to describe protein or collagen-based hard and soft tissues, from animal and human donors or created synthetically or artificially, including, but not limited to bone, cartilage, tendon, ligament, collagen scaffolds, collagen substrates, tissue meshes, cornea, and heart valves.

As used herein, the term “allotransplantation” is used throughout the specification to describe the transplantation of cells, tissues, or organs, to a recipient from a (genetically non-identical) donor of the same species. The transplant is called an allograft or allogeneic transplant or homograft. Most human tissue and organ transplants are allografts. In contrast, a transplant from another species is a xenograft. A transplanted organ or tissue from a genetically identical donor (such as an identical twin) is an isograft. When a tissue is transplanted from one site to another on the same patient, it is an autograft. In bone marrow transplantation, a genetically identical graft is syngeneic [13], whereas the equivalent of an autograft is termed autologous transplantation [14].

As used herein, the term “patient” or “subject” is used throughout the specification to describe an animal, generally a mammal and preferably a human, to whom treatment, including prophylactic treatment, with the compositions according to the present invention is provided. In certain embodiments, the patient or subject is a primate. Non-limiting examples of human subjects are adults, juveniles, infants and fetuses. For treatment of conditions or disease states, which are specific for a specific animal such as a human patient, the term patient refers to that specific animal.

As used herein, the terms “prevention” or “preventing” includes: (1) inhibiting the onset of a disease in a subject or patient which may be at risk and/or predisposed to the disease but does not yet experience or display any or all of the pathology or symptomatology of the disease, and/or (2) slowing the onset of the pathology or symptomatology of a disease in a subject or patient which may be at risk and/or predisposed to the disease but does not yet experience or display any or all of the pathology or symptomatology of the disease.

As used herein, the terms “treat” and “treating” is used throughout the specification to describe a step or several steps of a process to achieve a goal. In particular, most treatments considered include the process of sterilization by exposure to a genipin solution. Additionally, as used herein, the terms “treat” and “treating” are not limited to the case where the subject or material (e.g. tissue, substrate, or patient) is cured and the disease is eradicated or material sterilized. Rather, the present invention also relates to treatment that merely reduces symptoms, improves (to some degree) and/or delays disease progression. It is not intended that the present invention be limited to instances wherein a disease, infection, or affliction is cured. It is sufficient that symptoms are reduced.

As used herein, the terms “post-treat” and “post-treating” is used throughout the specification to describe a steps in a process subsequent to the initial tissue or substrate sterilization step.

As used herein, the terms “sterilization” is used throughout the specification to describe a term referring to any process that eliminates (removes) or kills all forms of microbial life, including transmissible agents (such as fungi, bacteria, viruses, spore forms, etc.) present on a surface, contained in a fluid, in a compound such as biological culture media, or tissue or substrate. The term “sterilization” also includes the disabling or destruction of infectious proteins such as prions.

The present invention relates to the above-described compositions in “therapeutically effective amounts” or “pharmaceutically effective amounts”, which means that amount which, when administered to tissues is sufficient to effect such treatment for the disease, infection, or to ameliorate one or more symptoms of a disease or condition (e.g. ameliorate pain).

As used herein, the term “salts”, as used herein, refers to any salt that complexes with identified compounds contained herein while retaining a desired function, e.g., biological activity. Examples of such salts include, but are not limited to, acid addition salts formed with inorganic acids (e.g. hydrochloric acid, hydrobromic acid, sulfuric acid, phosphoric acid, nitric acid, and the like), and salts formed with organic acids such as, but not limited to, acetic acid, oxalic acid, tartaric acid, succinic acid, malic acid, fumaric acid, maleic acid, ascorbic acid, benzoic acid, tannic acid, pamoic acid, alginic acid, polyglutamic, acid, naphthalene sulfonic acid, naphthalene disulfonic acid, and polygalacturonic acid. Pharmaceutically acceptable salts also include base addition salts, which may be formed when acidic protons present are capable of reacting with inorganic or organic bases. Suitable pharmaceutically-acceptable base addition salts include metallic salts, such as salts made from aluminum, calcium, lithium, magnesium, potassium, sodium and zinc, or salts made from organic bases including primary, secondary and tertiary amines, substituted amines including cyclic amines, such as caffeine, arginine, diethylamine, N-ethyl piperidine, histidine, glutamine, isopropylamine, lysine, morpholine, N-ethyl morpholine, piperazine, piperidine, triethylamine, trimethylamine. All of these salts may be prepared by conventional means from the corresponding compound of the invention by reacting, for example, the appropriate acid or base with the compound of the invention. Unless otherwise specifically stated, the present invention contemplates pharmaceutically acceptable salts of the considered pro-drugs.

As used herein, the term “effective,” as that term is used in the specification and/or claims, means adequate to accomplish a desired, or hoped for result.

As used herein, the term “hydrate” when used as a modifier to a compound means that the compound has less than one (e.g., hemihydrate), one (e.g., monohydrate), or more than one (e.g., dihydrate) water molecules associated with each compound molecule, such as in solid forms of the compound.

An “isomer” of a first compound is a separate compound in which each molecule contains the same constituent atoms as the first compound, but where the configuration of those atoms in three dimensions differs.

As used herein, “room temperature” or “RT” refers to approximately 25° C.

### DESCRIPTION OF THE FIGURES

The accompanying figures, which are incorporated into and form a part of the specification, illustrate several embodiments of the present invention and, together with the description, serve to explain the principles of the invention. The figures are only for the purpose of illustrating a preferred embodiment of the invention and are not to be construed as limiting the invention.

FIG. 1 shows the flow cytometry results of rat adipose derived stem cells on day 8 after cell culturing showed the CD29+ and CD90+ cells were the predominant cell type. Spindle-shaped morphological features of the stem cells are shown on the right on the 2^{nd} (top) and a 8^{th} (bottom) day after harvesting (200×).

FIG. 2 shows human adult dermofibroblasts seeded into fibrin glue survive and proliferate rapidly to 90% confluence in culture media in vitro within 7 days.

FIG. 3 shows a scatter plot of abdominal leak point pressures in control rats compared with rats undergoing pubourethral ligament transection (PULT) without treatment or with local treatment fibrin glue, rat adipose derived stem cells (rADSC), fibrin glue+rat ADSC, fibrin glue+human dermofibroblasts.

FIG. 4 shows sagittal cross-sections of the pubourethral ligamentarea, including the symphysis pubis, urethra and vagina stained with Mason Trichrome (40×; top) and Hematoxilin Eosin (H&E; middle). encapsulated rat adipose derived stem cells (ADSC) and dermofibroblast-treated groups demonstrated evidence of profound collagen deposition and several new micro vessel formations (white arrows) in the former pubourethral ligament transection (PULT) area also confirmed with CD31 immunofluorescence staining (40× or 100×; bottom); (Green arrows show the figure with higher magnification of the circled area under fluorescent microscope red filter). Moreover some animal's urethra in these 2 groups demonstrates a bridge formation between the urethra and the pubic bone.

FIG. 5 shows in the groups of pubourethral ligament transection (PULT)+rat adipose derived stem cells (rADSCs) in fibrin glue treatment, there was the most activity in the PULT space, involving sheaths of cells with the largest structure of adipose tissue with several numbers of newly formed microvasculature (on left) qualitatively different from adipose tissue which formed in the fibrin glue only and rADSC only treated groups (on right), in that the cell membranes had thick cytoplasm and more enhanced nucleus.

FIG. 6 shows the site of pubo-urethral ligament (PUL) in humans. PUL is a drape-like structure, fixing the ventral surface of the urethra to the dorsal surface of symphsyis pubis.

FIG. 7 shows the sagital views of PUL ventral to urethra in damaged conditions (A) and normal (B) conditions in female rat. Please note the separation of urethra from dorsal surface of the pubis.

FIG. 8 shows leak point pressure (LPP) measured in Female SD rats, 4 (A) and 28 days after PUL injury, sham or pudendal nerve transection (PNT).

FIG. 9 shows a transabdominal view of PUL in female canine. Surgical pick up is pushing down the urethra to demonstrate the drape-like attachments of PUL structure.

FIG. 10 shows supporting data for the current invention showing LLP results for PULT in a rat model system with rat adipose stem cells/glue/fibroblasts.

FIG. 11 shows that local administration of bone marrow derived human mesenchymal stem cells (hMSCs) and a dityramine cross-linked porcine collagen-based hydrogel (T-gel) could restore continence in a model of PUL deficiency.

### DETAILED DESCRIPTION OF THE INVENTION

### I. Introduction

The invention relates to methods for the treatment of urinary incontinence (UI) and stress urinary incontinence (SUI) in both men and women by replacement of a damaged, absent or injured pubo-urethral ligament (PUL) via a simple injection of “Regenerative Glue”. For example, in the case of UI, the injection of “Regenerative Glue” at the PUL site will adhere the ventral surface of the urethra to the dorsal surface of the symphysis pubis (i.e. the space of Retzius), and further provide regenerative material for continuous replacement of deficient tissues and cells. For example, this “Regenerative Glue” may replace and potentially regenerate the PUL and other ligaments in the pelvis: a) an adhesive element similar in composition to connective tissue or the PUL and other ligaments; and b) a regenerative element that can interact with the surrounding environment (such as fibroblasts, fibromyoblasts, collagen, etc) to refurnish damaged or absent elements of the PUL and other ligaments. “The Regenerative Glue” can be made from several commercially available materials including biocompatible glues, fibrin sealant or biocompatible gels; combined with Meschencymal stem cells (MSC) derived from bone marrow or adipose tissues.

One reference, Yamada, Y. et al. (2003) Bone Regeneration Following Injection of Mesenchymal Stem Cells and Fibrin Glue with a Biodegradable Scaffold, Journal of Cranio-Maxillofacial Surgery 31, 27-33 [15]. This reference discloses the use of a combination of fibrin glue, biodegradable β-tricalcium phosphate (β-TCP) and mesenchymal stem cells to provide three-dimensional templates for new bone formation at heterotopic sites in a rat model. Stem cells and developing matrices, explanted from the rat femur, were fragmented and mixed with β-TCP fibrin glue in a syringe. The cell/β-TCP fibrin glue admixtures were injected into the subcutaneous space on the dorsum of the rat. Eight weeks after implantation, gross morphology revealed a pearly opalescence and firm consistency. Histological inspections showed newly formed bone structures in all admixtures, but none in the control groups when only fibrin glue and β-TCP were injected. Osteopont, a protein that may be involved in bone development, was identified detecting antibodies present in all cell/β-TCP fibrin glue admixtures. The authors concluded that mesenchymal stem cell/β-TCP fibrin glue admixtures could result in successful bone formation and speculate that this technique represents a minimally invasive means of generating autogenous bone to correct or reconstruct bone defects. This reference does not disclose using such a technique to address SUI.

Another reference, Ahmed, T. A. E. et al. (2011) Fibrin Glues in Combination with Mesenchymal Stem Cells to Develop a Tissue-Engineered Cartilage Substitute, Tissue engineering. Part A 17, 323-335 [16]. This reference evaluates tissue engineering-based constructs to restore cartilage structure and function by mobilizing chondrogenic cells. Fresh fibrin (FG) and platelet-rich fibrin (PR-FG) glues produced by the CryoSeal FS System, in combination with human bone marrow-derived mesenchymal stem cells (BM-hMSCs), were evaluated. The authors concluded that FG is more promising than PR-FG as a scaffold for chondrogenic differentiation of hMSCs; however, immobilization of growth factors inside these fibrin scaffolds with the HBDS system had a negative impact. In addition, the authors contend that BM-hMSCs are valid and potentially superior alternatives than chondrocytes for tissue engineering of articular cartilage. The authors also suggest that MSC-based approaches would be suitable for constructs at the cartilage/bone interface and to develop ligament and meniscus substitutes. This reference does not disclose using such a technique to address SUI.

Another reference, Fang, H. et al. (2004) Biocompatibility Studies on Fibrin Glue Cultured with Bone Marrow Mesenchymal Stem Cells in Vitro, J. Huazhong Univ. Sci. Technolog. Med. Sci. 24, 272-274 [17]. This reference investigates the biocompatibility of fibrin glue materials for scaffolds in bone tissue engineering by culturing rabbit bone marrow mesenchymal stem cells with fibrin glue in vitro. Processed bone marrow mesenchymal stem cells grew on the surface of fibrin glue and adhered to it gradually. Fibrin glue had no inhibitory effect on cell morphology, growth, proliferation and differentiation. The authors concluded that the fibrin glue exhibited good biocompatibility and can be used as scaffold materials for bone tissue engineering. This reference does not disclose using such a technique to rebuild ligaments or to address SUI.

Another reference, Roche, R. et al. (2010) Stem Cells for Stress Urinary Incontinence: The Adipose Promise, J. Cell. Mol. Med. 14, 135-142 [18]. This review article discloses the potential use of muscle precursor cells (MPCs) for the treatment of UI. Numerous studies have shown the multipotent or even the pluripotent nature of the stromal vascular fraction (SVF) or adipose-derived stem cells (ASCs) from adipose tissue. These cells are capable of acquiring many different phenotypes in vitro. Furthermore, recent animal studies have highlighted the potential use of SVF cells or ASCs in cell therapy, in particular for mesodermal tissue repair and revascularization. Moreover, this reference discusses the potential interest of SVF cells or ASCs for the treatment of UI in women. The authors concluded that because access to these cells via lipoaspiration is simple, and because they are found in very large numbers in adipose tissue, their future potential as a stem cell reservoir for use in urethral or other types of cell therapy is enormous. This reference does not disclose using fibrin glue or mesenchymal stem cells to address stress UI via reconstruction of the PUL.

Another reference, Furuta, A. et al. (2007) Advances in the Understanding of Stress Urinary Incontinence and the Promise of Stem-Cell Therapy, Rev. Urol. 9, 106-112 [19]. This reference discloses that cell-based therapies using autologous multipotent stem cells procured from bone marrow may be painful, require anesthesia, and yield low numbers of mesenchymal stem cells upon processing. The authors report that muscle and adipose-derived stem cells can be obtained easily in large quantities under local anesthesia. The authors envision the restoration of function using autologous stem cells rather than lifting the urethra with a sling or bulking up the urethral sphincter with collagen. This reference does not disclose using fibrin glue and mesenchymal stem cells to address UI.

Another reference, Cheng, H. et al. “Fibrin Glue Composition for Repairing Nerve Damage and Methods Thereof,” United States Patent Application 20090191165 (published Jul. 30, 2009) [20] (herein incorporated by reference). This reference discloses a composition for repairing nerve damage and/or enhancing the functional recovery of a damaged nerve that includes fibrin glue and bone marrow stem cells (BMSCs). This reference also discloses a method for repairing nerve damage and/or enhancing the functional recovery of a damaged nerve by topically applying the fibrin glue. This reference does not disclose using such a technique to rebuild ligaments or to address stress UI.

Another reference, Motlagh, D. et al. “Using of Scaffold Comprising Fibrin for Delivery of Stem Cells,” United States Patent Application 20100028311 (published Feb. 4, 2010) [21] (herein incorporated by reference). This reference discloses the delivery of cells to desired tissue sites, prolonged retention of the cells at those sites, and the integration of cells into an area of interest for increased therapeutic effect. The reference further describes compositions and methods for treating ischemia in a subject. In some aspects, the methods of treatment comprise the administration of a fibrin scaffold or fibrin clot comprising stem cells. This reference does not disclose using such a technique to rebuild ligaments or to address stress UI.

Another reference, Milano, G et al. “Method of Arthroscopic Osteochondral Resurfacing Using PRP Strengthened with Fibrin Glue,” United States Patent Application 20090062870 (published Mar. 5, 2009) [22] (herein incorporated by reference). This reference discloses methods of arthroscopic resurfacing of a joint utilizing a biological component strengthened with fibrin glue. The biological component is selected from the group consisting of platelet-rich plasma (PRP), bone marrow aspirate (BMA) and autologous conditioned plasma (ACP). The biological component/fibrin glue composition may be inserted (by injection or by employing a biologic resurfacing mold) into a transosseous tunnel in the vicinity of the defect. Upon insertion at the defect site, the biological component/fibrin glue composition is designed to coagulate and solidify within a few minutes to advance the healing of the damaged tissue and tissue growth. The biological component/fibrin glue composition may optionally comprise components such as growth factors, antiseptic chemicals, antibiotics, electrolytes, hormones or site-specific hybrid proteins, among others. This reference does not disclose a combination of fibrin glue and stem cells to rebuild ligaments or to address SUI.

Another reference, Chancellor, M. B. et al. “Rapid Preparation of Stem Cell Matrices for Use in Tissue and Organ Treatment and Repair,” U.S. Pat. No. 7,906,110 (published Mar. 15, 2011) [23] (herein incorporated by reference). This reference discloses the preparation of stem cell and physiologically acceptable matrix compositions. The stem cell-matrix compositions of the present invention do not require long-term incubation or cultivation in vitro prior to use in vivo applications. The stem cells can be from numerous sources and may be homogeneous, heterogeneous, autologous, and/or allogeneic. The stem cell-matrix compositions provide point of service utility for the practitioner, wherein the stem cells and matrix can be combined not long before use, thereby alleviating costly and lengthy manufacturing procedures. In addition, the stem cells offer unique structural properties to the matrix composition, which improves outcome and healing after use. Use of stem cells obtained from muscle affords contractility to the matrix composition. The compositions of the invention can be used to treat diseases such as impaired muscle contractility of the heart, diaphragm, gastrointestinal tract, and genitourinary tract. The present invention is also made for a variety of treatments, repairs, augmentations, filling and healing of skin (dermis and epidermis) and soft tissue, muscle, bone, ligaments, so as to reduce scarring that results from conventional techniques. This reference does not disclose a combination of fibrin glue with stem cells to rebuild ligaments or to address SUI.

Another reference, Schuldt, A. J. T. et al. “Fibroblast Derived Stem Cells,” United States Patent Application 20110041857 (published Feb. 24, 2011) [24] (herein incorporated by reference). This reference discloses methods and compositions relating to the production of stem cells derived from de-differentiated fibroblasts, and the use of such stem cells for treatment of a variety of disorders and conditions. The invention is based on the discovery that a population of stem cells, capable of differentiating into a variety of different cell types, can be generated by culturing fibroblasts under selective culture conditions. This reference does not disclose using such a technique to rebuild ligaments or to address SUI.

Another reference, Fu, Y. “Treatment of Spinal Injuries Using Human Umbilical Mesenchymal Stem Cells,” United States Patent Application 20080305148 (published Dec. 11, 2008) [25] (herein incorporated by reference). This reference discloses that the transplantation of human umbilical mesenchymal stem cells (HUMSCs) to an area of a spinal injury is therapeutically effective in treating the spinal injury. Methods for treating spinal injuries based on such transplantation are described. This reference does not disclose using such a technique to rebuild ligaments or to address SUI.

Another reference, Lendeckel, S. et al. (2004) Autologous Stem Cells (Adipose) and Fibrin Glue Used to Treat Widespread Traumatic Calvarial Defects: Case Report, J. Craniomaxillofac. Surg. 32, 370-373 [26]. This reference discloses the use of autologous stem cells and fibrin glue to treat skull bone fractures. Combined with artificial materials, the stem cells (originally from the patient) and fibrin glue aided in healing the skull fracture. This reference does not disclose using such a technique to rebuild ligaments or to address SUI.

Urinary incontinence is one of the most prevalent conditions, affecting approximately 40% of women in the United States and the Western world. Stress urinary incontinence (SUI) accounts for the largest portion of these women. It is estimated that 30-40 percent of women, about the age of 60, have some level of SUI. SUI is the loss of urine associated with movements, such as coughing, sneezing, laughing, and exercise. It has significant impact on quality of life of women, and the aggregate social, medical and economic costs are massive. Moreover, the extrapolation from the US 2000 Census data on increase of proportion of women at high risk for development of SUI who would likely require treatment for SUI indicate that the size of this population will increase at least 2 to 3-fold within the next 1 or 2 decades [27]. The effective treatment of SUI mostly revolves around surgical treatments. Over the past several decades, several types of procedures have been proposed and frequently used [28]. The mid urethral sling procedures (MUS) have become the mainstay of surgical treatment of SUI in women. Specifically retro pubic Tension-Free vaginal Tape (TVT) is the most widely popular and used MUS procedure worldwide, with over 1 million procedures performed until now. It is estimated that over 250,000 procedures are done annually in the United State alone.

The mid-urethral slings were invented based on an ‘integral theory’ of pathophysiology of SUI, in which the deficient or weakened pubo-urethral ligament (PUL) cannot withstand the pressures imposed by cough, sneeze and other sources of increased intra-abdominal. It is proposed that PUL is damaged during pregnancy, vaginal delivery, and aging. Despite the common use of mid-urethral slings, use of this procedure is still associated with a disturbing level of complications including urinary retention, recurrence of SUI and urge urinary incontinence [29]. Thus, numerous investigators and industrial partners are in active search for a less invasive and morbid treatment of SUI.

Stem cell therapy and tissue engineering are among the newest strategies under investigation for the treatment and prevention of SUI. Several attempts have been made to develop such augmentations using different types of stem cell for the treatment of SUI. The first clinical series in the medical literature is with the use of muscle-derived cellular therapy by Carr et al. from Canada [30]. After about 17 months of follow-up, five patients out of eight remained in the study and got some improvement during follow-up, while one subject achieved total continence. The improvement in these subjects occurred between 3 and 8 months after the first injection. During follow-up, no serious adverse events were reported.

More clinical studies have been done evaluating SUI improvement after muscle derived stem cell and adipose derived stem cells (ADSCs) local injection into the urethra [18, 31-36]. A number of animal studies have been done on applying mesenchymal stem cells to improve SUI in animal models of urinary incontinence. All of them have demonstrated local application of MSCs in an animal model of SUI restored the damaged external urethral sphincter and significantly improved the SUI [37-39].

Adipose tissue has attracted interest as a possible alternative stem cell source to bone marrow. Enticing characteristics of ADSCs include: a) ease of extraction, b) higher content of mesenchymal stem cells (MSC) as compared to bone marrow, c) ex-vivo expandability of MSC in terms of differentiation ability, d) angiogenesis stimulating potential, and e) immune modulatory effects [39,40]. In double blind studies of canine osteoarthritis statistically significant improvements in lameness, range of motion, and overall quality of life have been described [41,42]]. The process of autologous fat grafting has been commonly used in cosmetic surgery [43], so, autologous cell therapy, with the numerous cellular populations besides MSC that are found in adipose tissue, is relatively easy-use and innocuous.

Zuk et al. demonstrated the SVF contains large numbers of mesenchymal stem cells (MSC)-like cells that could be induced to differentiate into adipogenic, chondrogenic, myogenic, and osteogenic lineages [44,45]. In addition to MSC content, it was identified that SVF contains endothelial precursor cells (EPC) and also monocytes/macrophages which possess anti-inflammatory functions [46]. Besides, numerous studies have demonstrated the ability of MSC to induce immune suppression [47].

### II. Adipose-Derived Stem Cells Plus Glue Restore Continence

Stress urinary incontinence (SUI) affects millions of women world-wide and has a significant impact on quality of life. It has been proposed that the pubourethral ligament (PUL) is damaged during pregnancy, vaginal delivery and aging, contributing to SUI. The objective of this study was to evaluate the potential for adipose-derived stem cells (ADSCs) encapsulated in fibrin glue to repair or regenerate the damaged or absent PUL in a rat model of SUI. Rat ADSCs (rADSCs) were harvested and cultured for up to 8 days. Cellular characteristics were determined via flow cytometry. PUL transaction (PULT) was performed in 120 female Sprague-Dawley rats and rats were then injected with either: i) 1.5 million rADSCs in fibrin glue, ii) 1.5 million rADSCs in 150 ml PBS, iii) 150 μl of fibrin glue, or iv) 1.5 million human dermofibroblasts in 150 μl fibrin glue, or no treatment. Continence status was assessed via leak point pressure (LPP) measurements and morphometric studies via histological exam of pelvic tissue with H&E and trichrome staining, or immunofluorescence using anti-CD31 antibody. rADSC seeded into fibrin glue survived and proliferated rapidly to 90% confluence in culture media. Mean LPP significantly improved in treated rats (P<0.05) compared with untreated rats, with best results obtained from rats treated rADSC+fibrin glue or human dermofibroblast+fibrin glue. Histopathology demonstrated regenerated adipose tissue in the PUL space after treatment with rADSC+fibrin glue. Overall, this study demonstrated the potential therapeutic benefit of autologous ADSCs in restoration of SUI.

### Introduction:

Prevalence data suggest that at some point, close to one-half of all U.S. women experience one or more manifestations of urinary incontinence (UI), with Stress Urinary Incontinence (SUI) being the predominant type [1,48] and one in 10 will undergo surgery for by the age of 80 [49]. Stress urinary incontinence (SUI), is defined as loss of urine associated with movements, such as coughing, sneezing, laughing and exercise. SUI has a significant impact on quality of life, and the aggregate social, medical and economic costs are massive [49-51].

The pathophysiology of SUI is described as an incurred defect resulting from damage to or weakening of the connections between fascia and muscle, the nerve supply, or the smooth or striated sphincters in the pelvic floor. This, in conjunction with loss of anterior vaginal wall support, may result in the failure of urinary resistance by the urethra, internal and external sphincter, and pelvic floor in response to increased intra-abdominal pressure. Over the past century several theories of pathophysiology of SUI have led to invention of therapeutic options including different types of surgery such as retropubic bladder neck suspensions, pubovaginal slings, or injection of bulking agents into the wall of urethra. The most recent of these theories (Integral Theory) developed by Dr. Petros, states that urinary leakage may be a result of a deficient pubourethral ligament (PUL) causing hypermobility of the urethra and subsequent diminished urinary resistance [52]. Mid-urethral slings (MUS) were developed based on the Integral Theory in early 1990s, and ever since have become the most common anti-SUI procedures performed around the world with an estimate of approximately 2 million procedures completed thus far. It is estimated that more than 250,000 procedures are performed annually in the United State alone [53-55].

The Integral Theory proposes that the PUL is damaged during pregnancy, vaginal delivery, and aging [29]. The placement of the mid-urethral sling is postulated to provide support to the damaged or deficient PUL. Given the wide popularity of Integral Theory and its MUS, several animal models of PUL deficiency [56,57] and MUS [58,59] for translational studies [60] have been created.

However, almost two decades of use of MUS in treatment of SUI indicates that similar to its preceding surgical procedures, the MUS has problems and cannot be considered as the ultimate anti-SUI treatment [61]. The problems with all invasive surgical anti-SUI including the MUS are two folds. First, failure or persistence of incontinence, when either the women continue to have problems with SUI or other forms of urinary incontinence. Second, complications, either short term such as bladder perforation, hemorrhage, bowel injury urinary retention, urge incontinence or long term such as difficulty in voiding, sling erosion, pain syndromes, de novo urgency and urge-associated incontinence, and urinary tract infections [29,62] Accumulatively, these two problems result in need for re-treatment in at least 30% of women with SUI [57, 61]. Thus, numerous investigators and industrial partners are actively searching for a less invasive and more efficacious treatments. Specifically, there is interest in finding a therapeutic approach that would have a restorative effect on the pelvic floor and urethral support mechanisms to prevent or reverse the imbalance between injury and recovery in SUI [63, 64].

Stem cell therapy has the potential to restore damaged tissues and perhaps improve the function of the organ affected by those damages. Mesenchymal stem cells (MSC) can act as agents of connective tissue homeostasis and repair by producing a paracrine effect on local tissues which modulates the immune response, recruiting and inducing mitosis of endogenous tissue progenitor cells, promoting angiogenesis, and preventing an inappropriate fibrotic response [65]. These cells can also exhibit a tissue-specific differentiation pattern and have been employed in a model targeting ischemic cardiomyopathy, where allogenic human MSC demonstrated differentiation into cardiomyocytes, vascular smooth muscle cells, and endothelial cells [65]. Several attempts have been made to develop such augmentations using muscle, bone marrow, cord blood and adipose-derived stem cells (ADSC) for the treatment of SUI [66-69].

Autologous rat ADSCs (rADSC) are an attractive cell source because of their (1) availability, (2) their high expansion, proliferative and differentiation potential, (3) their paracrine effects including immune modulation and angiogenesis stimulating potential, and, (4) simple aspiration procedures, which provide a direct translation into clinical trials. On the other hand, fibroblasts are believed to be one of the main cell types involved in the wound healing process [66]. Fibroblasts regulate extracellular matrix protein production and angiogenesis [66]. Several studies have also demonstrated a key role for fibroblasts and fibroblast-derived growth factors in the repair of injured ligaments [67]. However, cellular transplantation techniques are limited by transplanted cell maintenance and survival within the tissue. Degradable biomaterials have become an effective “scaffold” to fix and stabilize transplanted cells in other organs, including bone, cartilage, heart and skin [70]. Of the many biomaterials used for tissue engineering, fibrin matrix has favorable features similar to a scaffold, including biocompatibility, biodegradability, and binding capacity to the tissue. Furthermore, fibrin matrix is physiologically flexible, keeps seeded stem cells viable with intact characteristics, can facilitate cell growth and differentiation by its three-dimensional structure, has good plasticity and would fit well in the periurethral area of the pelvis [7]-74]. These features may be useful as a potentially suitable biological vehicle for cell transplantation therapy. Compared with other candidates for scaffolds, fibrin has the potential for simple extraction and production of patient's autologous fibrin with a low risk of foreign body reactions for patients. Fibrin glue is now approved by the FDA for multiple applications, including hemostasis in a broad variety of surgical specialties including colon sealing at the time of colostomy closure and skin graft adhesive attachment at the time of burn wound grafting. Stem cell implantation is one of the additional non-FDA-approved clinical uses of this material [75].

The aim of this study was to evaluate the feasibility and efficacy of use of autologous rADSC encapsulated in fibrin glue for the repair or regeneration of damaged or absent PUL, and whether such repair would improve the SUI in a rat model of PUL deficiency.

### Material and Methods:

### Animals

Female Sprague-Dawley breeder rats were used in this research, conducted under an approved protocol by the Institutional Animal Care and Use Committee, Case Western Reserve University, Cleveland, Ohio. ADSC were harvested from five rats (250-275 g, Harlan Laboratories, Indianapolis, Ind.) and 120 female retired breeder rats were used for the in situ functional study as described below. At the end of each study, animals were sacrificed under urethane anesthesia (1 g/kg, i.p.).

Harvesting and Culturing rADSC from Adipose Tissues:

Isolation of rADSC was performed through a method described by Zuk et al. [44] with a number of modifications. Briefly, subcutaneous adipose tissue was excised from the inguinal region and cut into fine pieces. The adipose tissue was rinsed with PBS containing 1% penicillin and streptomycin, minced into small pieces, and then incubated in a solution containing 0.1% collagenase type IA (Aldrich-Sigma, St. Louis, Mo.) for 1 hour at 37° C. while shaking vigorously. The digested tissue was centrifuged at 600 g for 10 min at room temperature. The pellet at the bottom of tube was then suspended in Dulbecco's-Modified Eagle's Medium (DMEM) supplemented with 10% fetal bovine serum (FBS, non-selected, Biomeda Corporation, Foster City, Calif.) and 10 ml of F-12 nutritional medial (DMEM:F-12, 1:1), filtered through a 100-μm cell strainer (BD Biosciences, Bedford, Mass.), and plated at a density of 1000 cells/mm2. A density of 1,000 cells/mm² was chosen as a high enough density for cell to cell contact but low enough to allow space for several days of proliferation without the cells becoming confluent. The culture dish was placed in a 5% CO₂ incubator for 8 days to allow the formation of MSC colonies, which were then trypsinized and propagated. During the 8 days of culture the media was changed after washing cells with PBS every 2-3 days.

Flowcytometry of rADSC:

Flowcytometric analysis was performed for identification of cellular characteristics after cell-labeling with appropriate antibodies 7 days after harvesting and culturing the cells. Flow cytometry was performed as described previously [76]. Antibodies directed against the following antigens were purchased from BD Pharmingen (San Diego, Calif.): CD45-FITC, CD29-PE, CD90-PE, CD271-PE. The cells were fixed in 500 μL of 1% paraformaldehyde (Electron Microscopy Sciences, Hatfield, Pa.). A minimum of 10,000 events were acquired per antibody set on a Becton Dickinson LSRII flow cytometer using Becton Dickinson FACS Diva software (version 6.2) (Becton Dickinson, San Diego, Calif.).

### Human Dermofibroblast:

Adult human dermal fibroblasts isolated from adult human skin were prepared from ScienCell Research Laboratories (Carlsbad, Calif.). They were cryopreserved at primary culture and delivered frozen. Each vial contained >5×10⁵ cells/ml. The dermofibroblasts were plated at a density of 1,000 cells/mm² in culture flasks and cultured in dermal fibroblast media (ScienCell) in a 5% CO₂ incubator. Human dermofibroblasts are characterized by their spindle morphology and immunofluorescent method with antibody to fibronectin.

### Fibrin Glue:

Fibrin glue is a two-component system that consists of human fibrinogen and human thrombin (Baxter Healthcare Corporation, Westlake Village, Calif.). When combined the two components mimic the final stage of the blood coagulation cascade.

### Viability Study of ADSCs in Fibrin Glue In Vitro:

Cultured rADSC were subsequently added to the thrombin component of fibrin glue at a concentration of 1 million cells per 200 μl thrombin. The thrombin and cell suspension was added to 200 μA of sealer protein in 75 cm² culture flasks. After 1 minute, 10 mL of culture media was added to the flask. After 1, 4 and 7 days, the proliferation of cultured cells were assessed under a microscope and was compared with the control rat ADSCs proliferation in media only.

### PUL Injury and Treatments:

Animals were anesthetized using inhaled isoflurane and then intraperitoneal injection with ketamine/xylazine. Analgesia was administered preoperatively with weight-based ketoprofen. All of the animals underwent PULT and then were allocated into 5 groups. PULT was performed by midline laparotomy, exposing the bladder neck, retracting the proximal urethra, and incising the visualized pubourethral ligament as previously described [57]. Immediately after PULT, rats were divided into 5 groups (n=24 each): 1) in situ (at the PULT site) injection of 150 μl of fibrin glue (75 μl thrombin and 75 μl of sealer protein cross-linked in situ), 2) in situ injection of 1.5 million rat ADSCs in 150 μl PBS, 3) in situ injection of 1.5 million rADSC in 150 μl fibrin glue (75 μl thrombin and rat ADSC suspension and 75 μl of fibrinogen (sealer protein) cross-linked in situ), 4) in situ injection of 1.5 million human dermofibroblast in 150 μl fibrin glue (75 μl thrombin and human dermofibroblast suspension and 75 μl of fibrinogen cross linked in situ), 5) negative control rats received no treatment. An additional 12 age-matched controls (positive control) did not undergo any PULT surgery and did not receive treatment.

### Functional Studies

Suprapubic tubes were implanted by reopening the midline incision twenty-six days after initial surgery with or without treatment. Two days later, leak point pressure (LPP) was measured in animals as has been previously reported [60]. A mean of 9-10 LPP measurements was calculated for each rat, and the difference between the means of each group was determined using a two-tailed unpaired t-test, assuming equal variance.

### Histopathology:

The whole pubic of animal was harvested and placed in Calrite® (a combination formalin/formic acid mixture working as fixative/decalcifier) for three days and then transferred to 70% alcohol solution. The region of interest was sectioned to a 2×2 cm block for paraffin embedding. The tissue specimen was dissected posteriorly between the vagina and rectum, laterally cut through the hip-femoral joints, and anteriorly up to the coronal plane even with the bladder dome. Finally the 2×2 cm pelvic block was cut 1 cm caudal to the urinary meatus in the axial plane in order to have the pubic bone, urethra and vagina consistently detectable in the plane. Cross sections were cut along this plane to prepare slides. H&E and trichrome stained slides from each group were examined under alight microscope. The area of focus was the urethra and the periurethral space, specially the site of the PULT, which is the area between the urethra and pubic bone, named as space of Retzius in human. The following parameters were compared among each group: edema and inflammation on H&E stained slides, tissue regeneration, collagen deposition and vasculature pattern on trichrome stained slides. Vasculature of the tissue surrounding the urethra is scored as normal or increased. To confirm the vasculature status the sections were stained for primary anti-CD31 antibody (Santa Cruz Biotechnology, Calif.) followed by staining with Alexa Fluor-568 secondary antibody (Invitrogen, OR) and imaged under immunofluorescent microscope.

### Results:

Fate of Stem Cells—

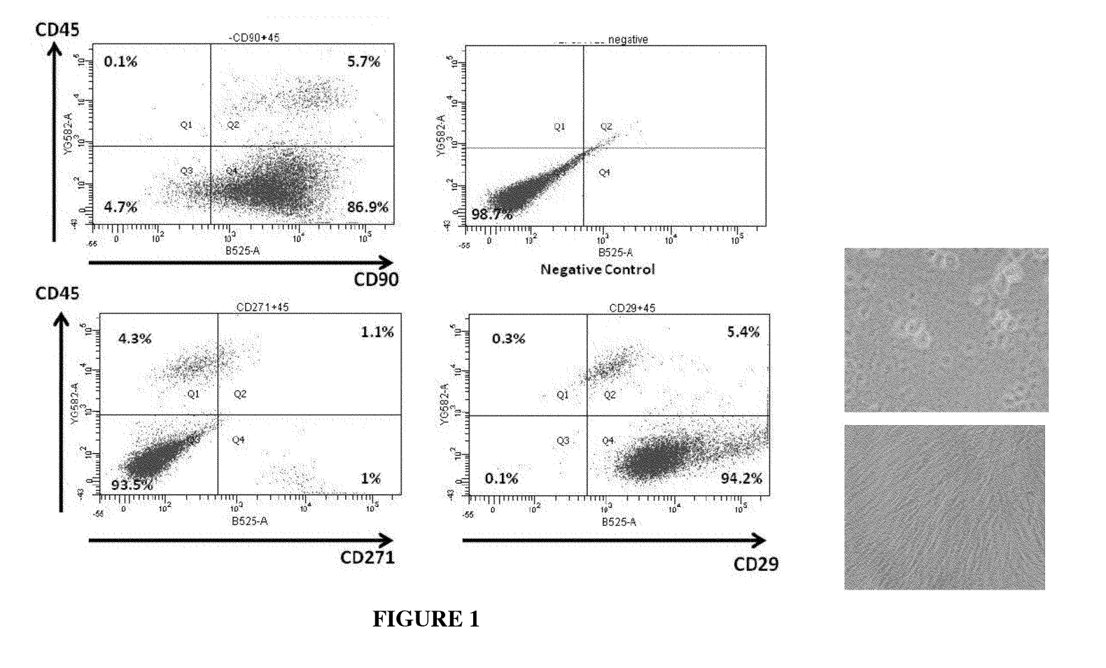

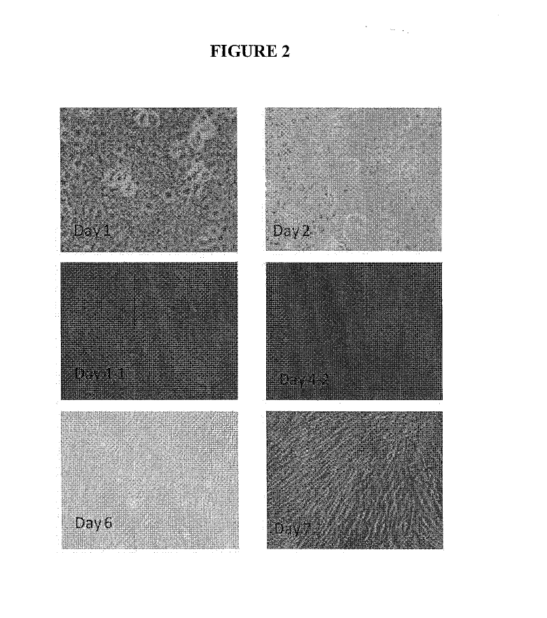

Primary ADSC cell cultures were attached after 24 hours, and reached 80% confluence after 7 to 9 days. The shape of single cells was a short fusiform with clear, elliptical nuclei. Uniform cell morphology and clustered cell growth were also observed. After 8 days and right before harvesting for injection, the cells appeared to maintain said shape, and were distributed evenly with uniform morphology (FIG. 1). Flow cytometry results of rADSC, 8 days after cell culturing showed the CD29+ and CD90+ cells were high in the population of stem cells. Less than 0.5% of the cells were positive for CD45 marker (FIG. 1). rADSC seeded into fibrin glue survived and proliferated rapidly to 90% confluence in culture media in vitro within 7 to 8 days (FIG. 2).

Urinary Incontinence—

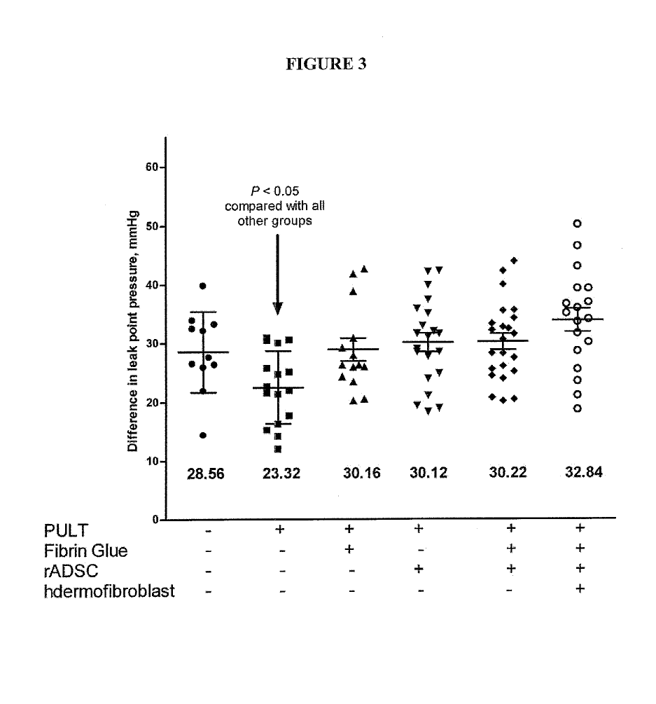

The mean LPP decreased significantly in PULT alone group compared with controls (no manipulations) one month after surgery (mean LPP 23.32 cm H₂O vs. 28.56 cm H₂O, respectively, p<0.05) (FIG. 3). Among the treated groups, there was a similar trend of improvement in the mean LPP values, including fibrin glue, rADSC+fibrin glue, and human dermofibroblast+fibringlue (FIG. 3). All treatment groups demonstrated a significantly greater mean LPP compared with the untreated PULT group (p<0.05) but not compared with the control group (p>0.05). The mean LPP in the human dermofibroblast+fibrin glue treatment group was 32.84 cm H₂O, which was higher but not significantly greater than the control or other treatment groups (p>0.05).

Histopathology—

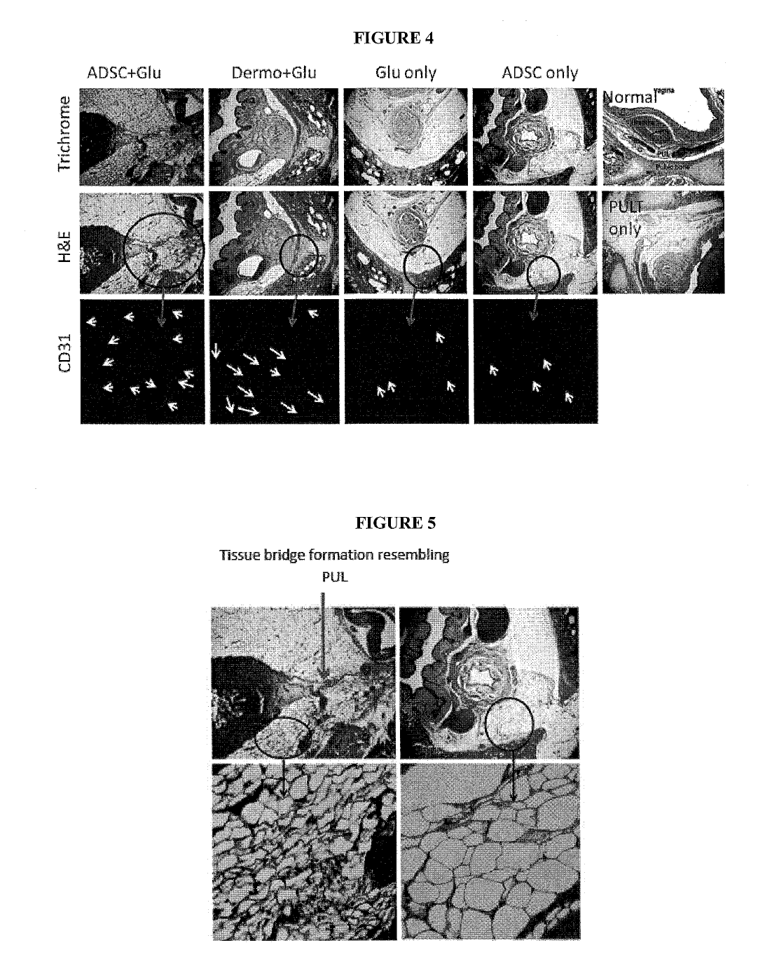

Sagittal cross-sections of the PUL area, including the symphysis pubis, urethra and vagina were stained with Hematoxilin Eosin (H&E) and Mason Trichrome (FIG. 4). Negative control PULT animals showed haphazard discrete, disorganized tissue, without much evidence of tissue regeneration (FIG. 4). In PULT+fibrin glue group animals, the site of the PULT space showed no active tissue formation or reaction. It appeared that the glue is partially absorbed after 28 days post-PULT, when the sections were made. In some of the cases a mature adipose tissue was seen inpart of the space. In the group of PULT+fibroblast+fibrin glue, most of the midurethral sections showed adhesion of the urethra behind the symphysis pubis with formation of granulation tissue. There was also evidence of new vascularization in this area which was confirmed with antiCD31 antibody staining (FIG. 4).

There was the most activity in the PULT space, involving sheaths of cells with the largest structure of adipose tissue with several numbers of newly formed microvasculaturein the group of PULT+rADSC+fibrin glue treatment. However, this adipose tissue was qualitatively different from typical adipose tissue (which formed in the previous fibrin glue only and ADSC only treated groups), in that the cell membranes had thick cytoplasm and more prominent nucleus (FIG. 5). Most importantly, in part of this adipose tissue, there appear to be a transformation occurring in the cytoplasm that is stained with red, very similar to myofibrins and collagen in other tissues. In the dermofibroblast treatment group, the tissue regeneration has more granulation tissue characteristics, whereas the rADSC treatment group has more adipose tissue differentiating into myofibrin-containing cells (FIG. 5). In addition, both encapsulated rADSC and dermofibroblast-treated groups demonstrated evidence of profound collagen deposition and several new micro vessel formations in the former PULT area also confirmed with CD31 immunoflourescence staining. Moreover some animal's urethra in these 2 groups demonstrates a tissue bridge formation between the urethra and the pubic bone, resembling a loose PUL structure (FIG. 4). It appears that the group treated with rADSC alone, showed similar results to the ones treated with fibrin glue only, in that there is only mature adipose tissue and few newly formed micro vessels in the former PULT area.

Additionally, both encapsulated rADSC and dermofibroblast treated groups demonstrated evidence of profound collagen deposition and formation of several new micro vessels in the former PULT area, which was confirmed with CD31 immunofluorescent staining.

### Discussion

SUI accounts for the largest portion of women in the United States and western world affiliated with urinary incontinence. Moreover, extrapolating from the US 2000 Census data on the increasing proportion of women at high risk for development of SUI, indicates that the size of the population requiring treatment will increase at least 2 to 3-fold within the next two decades [49, 50].

In previous preclinical experiments on stem cell therapy for SUI, incontinence is typically induced by sphincter injury, nerve injury, or vagina distension. The PULT animal model for SUI is based on the integral theory [57]. It has been shown that PULT and pudendal nerve transaction (PNT) produce equivalent results in terms of LPP in a SUI model [57] while PULT represents a novel animal model of periurethral connective tissues and ligaments weakness and instability concentrating on anatomical aspects of SUI.

There are several concerns with the currently available treatments of SUI, including failures and bothersome complications, resulting in approximately 30% re-do or undo of the treatments [77-79]. Among alternative treatments, stem cell therapy, which has the potential to restore continence naturally, is presently considered to have the best prospect to succeed.

Several attempts have been made to develop such augmentations using different types of stem cells for the treatment of SUI. The initial experiments were developed based on injection of skeletal myoblasts and later other types of skeletal muscle-derived cells into the weakened urethra to replenish the sphincter muscle [80]. Since Yiou et al. published the first preclinical study in 2002, applying skeletal muscle precursor cells to treat a murine model of urethral sphincter injury [68], several additional preclinical and clinical studies have been published applying skeletal muscle-derived cells, bone marrow stem cells, umbilical cord blood stem cells and adipose-derived stem cells (ADSC) [33, 69, 81, 82]. Almost all of these preclinical and clinical studies have demonstrated the efficacy of stem cells with different levels of SUI improvement.

In order to translate stem cell therapeutic approaches to the clinical setting, non-invasive methods for the application and development of stem cells must be developed. Since human adipose tissue is ubiquitous and easily obtainable in large quantities under local anesthesia with little patient discomfort, it presents an appealing source of stem cells for mesenchymal tissue regeneration and engineering. De Ugarte et al., suggests that there is little difference between cells derived from marrow and fat in terms of yield, growth kinetics, cell senescence, multi-lineage differentiation capacity, and gene transduction efficiency [83]. Moreover, in a study by Little, et al., a novel ligament-derived matrix could enhance expression of a ligamentous phenotype in hASC, within 28 days, in vitro [84]. In this study, an evaluation of rADSC as a modality of SUI cell therapy in PULT rats was desired.

The first published study demonstrating the efficacy of ADSC in treating SUI in a rat model was performed by Lin, et al. [85]. They found that transplantation of ADSCs via urethral or intravenous injection was effective in the treatment and/or prevention of SUI in a preclinical setting of vaginally distended animals. A clinical investigation on the efficacy of ADSC intreating post-prostatectomy SUI in 2 patients was published in 2010; however this was later withdrawn [35]. Several more groups have also demonstrated the value of ADSC in treating SUI [36, 69, 86]. Intraurethral injection of ADSC into a SUI rat model has been shown to improve the urethral connective tissue [85], and it is proposed that this is due to the ability of ADSC to generate and process collagen and elastin [86]. The integration of cell-scaffolding constructs represents a further advancement in the application of stem cells by providing a homing support for three-dimensional tissue growth and differentiation. These constructs are biocompatible, stable, promote growth of native tissues, and, ideally, will eventually become replaced with healthy, functional tissue.

Tissue engineering approaches, which combine the use of an appropriate cell source and biocompatible and biodegradable scaffolds controlling the release of growth factors, have been widely employed to restore the function of damaged tissue, including bone, cartilage, and neurons [87]. Zhao, et al. [88] demonstrated controlled-release nerve growth factor (NGF) by PLGA-loading NGF could significantly improve the therapeutic efficacy of ADSC in SUI rats. Fibrin glue is a unique FDA-approved surgical hemostatic/adhesive material that is being utilized with increasing frequency in a variety of surgical and grafting situations. The off-label sealing and adhesive applications are utilized by a wide variety of specialties and extend from fistula closure and seroma prevention to mesh fixation and stem cell implantation [75, 89]. Baumgartner, et al. demonstrated that human MSCs seeded into large-holed porous preparations of fibrin glue survive, proliferate and keep their stem cell characteristics [72]. Zhang, et al. studied transplantation of ADSC with injectable fibrin scaffolds [71]. ADSC plated in fibrin glue onto four-well culture plates underwent LIVE/DEAD staining, which demonstrated that ADSC survived and proliferated well in fibrin glue. They also showed transplantation of cells with injectable fibrin scaffold could preserve cardiac function in infarcted rat hearts [71]. These results are similar to these findings in which the rADMSC survived and proliferated in the fibrin glue. Garcia-olmo, et al. demonstrated the efficacy of expanded ADSC in combination with fibrin glue for the treatment of complex perianal fistula in a phase II clinical trial [90]. Another study showed that delivering uncultured marrow mononuclear cells within fibrin glue hydrogels to porous scaffolds could enhance bone formation within critical-sized rat cranial defects [91]. Fibrin glue, in practice, is a two-component system in which a solution of concentrated fibrinogen and factor XIII are combined with a solution of thrombin and calcium in order to form a coagulum, simulating the final stage of the clotting cascade. Once the thrombin/calcium is combined with the fibrinogen/factor XIII, a fibrin clot forms in seconds, or somewhat slower if a more dilute form of thrombin is used. Additionally, fibrin glue has been shown to be associated with more continuous cell growth and enhanced cellular organization, in vitro [92]. Becker, et al. showed fibrin glue leads to increased proliferation of fibroblasts and local accumulation of vascular endothelial growth factor (VEGF) [93].

Fibrin glue was chosen as a carrier scaffold for the rADSC in this study because (1) upon local delivery it attaches to the site of the injured ligament and therefore undergoes shaping within the defected area instead of moving into the abdominal cavity, (2) has the ability to temporarily replace PUL function by attaching the urethra to the pubic bone before the stem cells can regenerate the defective tissue, (3) its ability to provide a homing for the stem cell survival and differentiation [94], (4) it has the ability to enhance the functional activity of cells [95], and (5) autologous fibrin glue can be prepared or the safe translation into clinical trials [96].

These invitro results demonstrated that rADSC seeded in fibrin glue survived and proliferated rapidly, similar to the in vitro proliferation rate of non-capsulated ADSC. As far as it is known, this is the first study that aimed to evaluate the efficacy of a combination treatment of rADSC with or without encapsulation in a fibrin glue scaffold in rats that have undergone PULT. The LPP results suggest that rADSC, encapsulated in fibrin glue ex vivo and injected at the site of PUL injury, restore the continence mechanism in this rat model of SUI. Additionally, the results demonstrate that human dermofibroblasts encapsulated in fibrin glue, rADSC alone, as well as fibrin glue alone can restore the LPP in PULT animals. Whether in long term, dermofiroblasts functional results will equal to those of rADSC will require studies with longer duration of follow ups.

The histopathology results in the fibrin glue only treated group demonstrated that fibrin glue was partially absorbed with very little to no residual foreign body reactions in the former PULT area. This suggests that the use of glue as a cell carrier is safe and non-reactive to the tissue. On the other hand, although the fibrin glue alone could restore the continence mechanism based on functional LPP data, there was not much evidence of granulation tissue, new vascularization nor collagen deposition visible 28^{th} days after treatment. This suggests that the glue/stickiness effects of fibrin glue in attaching the urethra to the pubic bone could be a cause of SUI improvement.

The combination of either the dermofibroblast or rADSC with fibrin glue provides a means for some type of tissue regeneration. This new angiogenesis pattern is strong evidence for the presence of growth factors affecting the restoration of structures responsible for SUI (FIG. 4 and FIG. 5).

Over all, these findings would suggest that the cell therapy (in this case dermofibroblast and rat ADSC) would require a carrier, so as to allow them to survive, accumulate, slowly release their secretions, better replicate, organize and maybe differentiate. In this study, each and every one of these effects could have led to the formation of either the granulation tissue or the semi-differentiated adipose tissue. These results suggest that observations should be repeated over a longer duration so that future levels of maturation and regeneration of tissue in the cell-based therapy could be evaluated. It appears that the groups treated with fibrin glue and dermofibroblasts, as well as fibrin glue and rADSC result in active regeneration of tissue. The main difference being that the dermofibroblast-regenerated tissue primarily consists of granulation type tissue with no structures mostly formed of cigar shaped fibroblast deposits of the collagen. Whereas in the group treated with the glue and the rADSCs, organized formation of what appears to be adipose tissue with rich cytoplasm, indicative of the process of differentiation into other tissues, such as smooth muscle or myofibrin containing tissue.

Both groups with encapsulated and non-encapsulated rADSC-treated showed minimal evidence of the presence of inflammatory cells, while the dermofibroblast-treated groups showed significant numbers of macrophages, specifically in periosteal layer attached to the pubic bone and around the urethra. This evidence of anti-inflammatory and immunomodulatory effects of ADSCs indicates an advantage of the use of ADSCs instead of dermofibroblasts.

There are several limitations to this study. First, the number of cells used to treat each animal was selected based on the use of stem cells in previous models of tissue injury and repair in other organ systems [81]. Dose-response effects for different concentrations of either rADSC or fibrin glue were not studied. Therefore, although either treatment alone produced partial but statically insignificant restoration of continence, it is not clear whether different doses of ADSC or fibrin glue would produce a different or more dramatic result. Second, injury induced by PULT and most other animal models of SUI are acute models, whereas most human clinical SUI is chronic condition. Four weeks in rat's life expectancy roughly represents 3-4 years of life span in human, so the selected time point of 28 days represents a reasonable time span in natural history of SUI. However, longer term studies may be needed. Thirds, all the major healing steps including fibroblast activity, monocyte activity, epithelial cell migration and proliferation, angiogenesis, collagen and collagenase synthesis are associated with growth factors such as fibroblast growth factor, platelet-derived growth factor, transforming growth factor β, tumor necrosis factor, matrix metalloproteinase, and VEGF, which are either being secreted by MSCs, or can be promoted by MSCs to be secreted from other cells. Further exploration of these growth factors is essential for determination of the mechanism of SUI improvement by MSCs. Cells were not labeled for tracking in this study, to avoid the possibility of changes in their characteristic from the labeling medium.

Conclusion: herein is demonstrated functional improvement and tissue regeneration in the PULT model of SUI by use of autologous rADSC encapsulated in fibrin glue. These findings demonstrated the potential therapeutic benefit of autologous ADSC in subjects with SUI.

### III. Autologous Human Adipose Tissue Stromal Vascular Fraction

In some embodiments, the methods of the present invention provide autologous human adipose tissue stromal vascular fraction (SVF) based on advantages including, but not limited to: (1) availability, (2) their high proliferative potential, (3) easy aspiration procedures on the patient who receives the cells and (4) their anti-inflammatory and paracrine effect, (5) capability performing the liposuction, SVF extraction and urethral injection in the same operative set up [97, 98].

Furthermore, non-expanded (non-cultured) SVF may also be used which means that the harvested stromal vascular fraction of fat tissue has not passed through any manipulation, cell extraction, purification and culture process and it is going to be used freshly. The advantage of using non-expanded SVF is the sheer number of stem cell available in the fresh SVF, not needing to be cultured in a laboratory over days in order to get the desired number of ADSCs to achieve what is called “therapeutic threshold.” The possibility of having enough number of cells without culture expanding the SVF reduces any risk of non-desirable differentiation of cells while expanded, and excludes the unknown effect of culture media, fetal bovine serum and trypsinization as parts of regular culture methods [99].

In one embodiment, the present invention contemplates treating UI by replacement of damaged or injured pubo-urethral ligament (PUL) via combination of a liquid carrier (scaffold) and mesenchymal stem cell that can repair or regenerate the damaged or absent PUL, or other damaged ligamentous structures of the pelvic, in both women and men. In preparation for the clinical trial, animal model of PUL deficiency has been developed [56,57]; efficacy of such repair has been demonstrated.

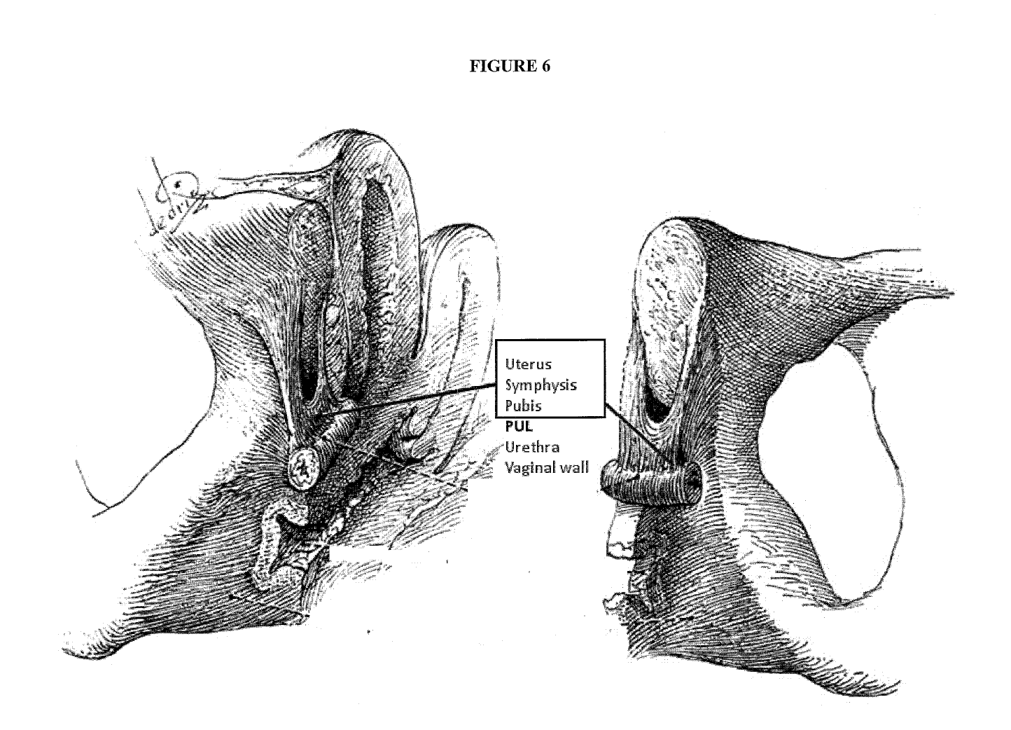

In one embodiment, the treatment of SUI by replacement of an injured PUL via simple injection of a biological glue that can replace the absent PUL by adhering the ventral surface of the urethra to the dorsal surface of the symphysis pubis (the so-called space of Retzius—the site of PUL; FIG. 6). As mentioned earlier, an animal model of PUL injury [56,57], in which SUI is generated in a reproducible fashion, has been developed.

One Example of PUL Therapy—

Although several possible combination of materials are compatible with PUL Therapy, autologous human adipose tissue stromal vascular fraction (SVF) plus Fibrin sealant (Baxter Healthcare Corporation, Westlake Village, Calif.) has been observed to have advantageous and superior results to conventionally used materials when treating damaged PUL by direct in-situ injection at peri-urethral site. For example, one such material comprises an FDA approved fibrin sealant (Baxter Healthcare Corporation, Westlake Village, Calif.). See, Example 1. Autologous human adipose tissue stromal vascular fraction (SVF) is a useful source of ADMSC that does not require IND recognition. Further, it should be noted that the peri-urethral site of injection has been commonly used in previous clinical situations for injection of bulking agents into or around urethra [100].

### Therapeutic Administration:

The subject will receive two sets of thrombin+SVF suspension (2 ml) and fibrinogen (sealer protein) component (2 ml) through Duploject applicator simultaneously as a treatment vial in order to repair/regenerate the pubo-urethral ligament defect.

One embodiment of a method for extracting, providing and administering the treatment vial is detailed in the Example 1. In brief, the whole process of liposuction, SVF extraction and injection takes approximately 2 hours. Moreover, all these procedures will be performed under aseptic conditions.

Conventional procedures of liposuction to extract abdominal fat will be performed in the operating room. Briefly, after performing local anesthesia and administrating vasoconstrictor (while the fat tissue settles and becomes less swollen), a stab wound in abdomen will be made by a 11 blade scalpel and stainless steel cannulas or microcannulas will be inserted into subcutaneous tissue. Then 200 to 300 mL adipose tissue is pumped out and collected in a sterile 1 L storage bottles (Corning, N.Y., USA).

It is notable that, the SVF harvesting protocol, which has been used previously, is based on the frequently applied methods by various research teams including [44, 101, 102], but with some modifications. After obtaining the fat tissue, it will be placed into 50-mL tubes with an equal volume of pre-warmed (37° C.) sterile phosphate buffered saline (PBS 1×) and agitated for 45 s. The lip aspirate is then washed to remove the majority of the erythrocytes and leukocytes.

Then, dispersion of adipose tissue is achieved by collagenase digestion. Collagenase has the advantage over other tissue digestive enzymes that it can efficiently disperse adipose tissue while maintaining high cell viability. At the end of this stage, patient's serum is added to stop collagenase activity. Following digestion, the ability of lipid-filled adipocytes to float is used to separate them from the stromal vascular fraction (SVF). This will be done by dispensing the collagenase-digested tissue into 50 ml tubes and centrifuge the suspension at 600×g for 10 minute at room temperature [103, 104].

Fibrin Glue is a two-component fibrin sealant that consists of human fibrinogen and human thrombin (Baxter Healthcare Corporation, Westlake Village, Calif.). When combined, the two components mimic the final stage of the blood coagulation cascade. In order to encapsulate the SVF in fibrin glue, prepare for injection, 2 sets of total 4 ml fibrin glue, and mix each 0.2 ml SVF suspension with one of the 4 ml kit were picked.

Afterward both components will be drained into two different 2 ml syringes and place them into the Duploject applicator (double-barrel 2 ml syringes connected by a Y-junction for mixing while injecting) set (Baxter Healthcare Corporation, Westlake Village, Calif.). Since there are two sets of SVF and glue, there will be a total volume of 8 ml including two thrombin+SVF suspension (2 ml) syringes, and two fibrinogen (sealer protein) component (2 ml) syringes. This treatment vial will be injected over the PUL defect field at the same time by Duploject applicator.

Based on pre-clinical data, improvement of SUI symptoms following PUL Therapy is expected to be observed. If proven true, the recruited patients may be among the first women to benefit from cell-based therapy without undergoing invasive surgical intervention.

Thus, specific compositions and methods of treatment of urinary incontinence with regenerative glue have been disclosed. It should be apparent, however, to those skilled in the art that many more modifications besides those already described are possible without departing from the inventive concepts herein. The inventive subject matter, therefore, is not to be restricted except in the spirit of the disclosure. Moreover, in interpreting the disclosure, all terms should be interpreted in the broadest possible manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided may be different from the actual publication dates, which may need to be independently confirmed.

### EXPERIMENTAL

### Example 1

### Stromal Vascular Fraction

The stromal vascular fraction (SVF) of adipose tissue is known to contain mesenchymal stem cells (MSC), T regulatory cells, endothelial precursor cells, preadipocytes, as well as anti-inflammatory M2 macrophages [104]. Accordingly, the SVF also contains blood cells from the capillaries supplying the fat cells. Safety of autologous adipose tissue implantation is supported by extensive use of this procedure in cosmetic surgery, as well as by ongoing studies using in vitro expanded adipose derived MSC (ADSC). Equine and canine studies demonstrating anti-inflammatory and regenerative effects of non-expanded SVF cells (non-cultured) have yielded promising results. Non-expanded SVF cells have been used successfully in accelerating healing of Crohn's fistulas and in treatment of multiple sclerosis [104]. Additionally, autologous SVF administration has been used commercially in over 3000 race horses for post-injury acceleration of healing, with published efficacy data in a double-blind canine osteoarthritis trial [41]. Currently autologous SVF is in clinical trials for post infarct remodeling [105], ischemic heart failure [106], type I diabetes [107], and liver failure [108].

The advantage of using non-expanded SVF is the sheer number of stem cell available in the fresh SVF, not needing to be cultured in a laboratory over days in order to get the desired number of ADSCs to achieve what is called “therapeutic threshold”. The possibility of having enough number of cells without culture expanding the SVF reduces any risk of non-desirable differentiation of cells when expanded and excludes the un-known effect of culture media, fetal bovine serum and trypsinization as parts of regular culture methods.

Side effect: Collagenase is an enzyme used here to extract the SVF from fat tissue. Because residual collagenase in prepared cell suspension for injection would cause probable local irritation in patient's tissue and it would have probable chemical contaminations [109], a “sterile” form of enzyme for excluding contamination probabilities is preferably used. Also the extracted SVF was washed and spun down several times to avoid having residual collagenase in the suspension and also the extracted SVF was treated with patient's blood serum to neutralize any potential residual collagenase enzyme [99].

The risk profile of stem cell based medicinal products depends on type of stem cell, their differentiation status and proliferation capacity, the route of administration, in vitro culture/manipulations step, immune responses. Herein, one advantage of using an autologous SVF excludes the HLA immune response. Avoiding culture expansion of cells avoids the hazards potentially associated with in-vitro expansion. SVF include mesenchymal stem cell populations (MSC) and the vast majority of small-sized clinical trials conducted with MSC in regenerative medicine applications has not reported major health concerns, suggesting that MSC therapies could be relatively safe. SVF proposes a potential risk of further MSC differentiation. The available literature indicates that such differentiation would follow the environments of the injected site by the cell-cell signaling interactions. As such, differentiation into smooth muscle cells, connective tissue fibroblasts, local increase in vascular supply, etc may be expected. Such differentiation would benefit the patients for repair of their damaged PUL. Patients with active malignancies or risk of further growth of malignant cells would be excluded.

### Example 2

### The Method for Extracting, Providing and Administrating the SVF

It is notable that the whole process of liposuction, SVF extraction and injection could take approximately 2 hours.

a—Liposuction

Conventional procedure of liposuction to extract abdominal fat will be performed at the operating room. In brief, after performing local anesthesia and administrating vasoconstrictor (while the fat tissue settles and becomes less swollen), an stab wound in abdomen will be made by a 11 blade scalpel and stainless steel cannulas or microcannulas will be inserted into subcutaneous tissue. Then 200 to 300 mL adipose tissue is pumped out and collected in a sterile 1 L storage bottles (Corning, N.Y., USA). The aspirate bottle will be kept in the ice packed box. For optimal results, only aspirate that has been obtained by tumescent liposuction method was used.

b—Adipose Tissue Preservation and Delivery:
The SVF harvesting protocol which is used is based on the frequently applied methods by various research teams including [44, 101, 102], but with some modifications.
The lipoaspirate should be processed in the biosafety cabinet (including a suction pipe) at room temperature in less than 1.5 hours.
   c—Reagent and Instrument Requirements
Sterile Phosphate Buffered Saline (PBS) (1×) 0.0067M (PO4) without phenol red; cat. no. 30256.02, Thermo Scientific).
Patients blood (100 cc) to obtain blood serum (50 cc)
Collagenase A type I (cat. no. C-0130, Sigma-Aldrich).
50 ml and 15 ml sterile plastic conical tubes (BD bioscience, Bedford, Mass.).
100-μm cell strainer (BD Biosciences, Bedford, Mass.)
50 ml, 25 ml and 10 ml sterile disposable plastic pipettes (BD bioscience, Bedford, Mass.).
A swing-out centrifuge with buckets for 50 ml and 15 ml tubes.
Shaker-incubator (37′C)
25 mm syringe filter, 022 um, MCE, sterile, Fisherbrand, Ireland.
Hemocytometer slide, trypan blue, light microscope for cell counting.
Fibrin sealant 4 ml kit including sterile DUPLOJECT applicator (double-barrel 2 ml syringes connected by a Y-junction for mixing while injecting) set (Baxter Healthcare Corporation, Westlake Village, Calif.)
Fibrinotherm heating and stirring device for preparing the Fibrin sealant (Baxter Healthcare Corporation, Westlake Village, Calif.)
175 cm2 sterile culture flask (to be used for collagenase treatment phase)
sterile Ringer-lactate solution
   d-SVF Separation and Suspension in Glue

### Phase 1:

Aseptically perform the steps below in a bio-safety cabinet, while wearing appropriate personal protective equipment:

Place tissue into 50-mL tubes with an equal volume of pre-warmed (37° C.) PBS and agitate for 45 s.

### d.1—Lipoaspirate Washing

It is necessary to wash the lipoaspirate extensively to remove the majority of the erythrocytes and leukocytes. The following procedures will be performed under aseptic conditions. All the tissue and cell process will be performed continuously within 1.5 hours at room temperature.

1. Allow the adipose tissue to settle above the blood/PBS fraction in 50-mL tube. This will take 1 to 2 minutes depending on the sample.

2. Carefully remove the lower phase using a 50 ml pipette.

3. Repeat the above washing procedure for three times.

4. Medium from the final wash should be clear. If it is still red, wash again by repeating the above steps.

Note:

As mentioned all these steps and following steps will be continuously performed as soon as possible after aspiration.

### d.2-Collagenase Digestion

Dispersion of adipose tissue is achieved by collagenase digestion. Collagenase has the advantage over other tissue digestive enzymes that it can efficiently disperse adipose tissue while maintaining high cell viability.

1. Make up collagenase solution just prior to digestion. The final volume required is half that of the washed adipose tissue volume. Add powdered collagenase to PBS at a final concentration of 0.05%. The required amount of collagenase was dissolved into 50 ml PBS, then filter sterilize into the remaining working volume (by 25 mm syringe filter, 0.22 um, MCE, sterile, Fisher brand).

2. Add the washed adipose tissue to large cell culture flasks (100 ml per 175 cm2 flask).

3. Add collagenase solution.

4. Resuspend the adipose tissue by shaking the flasks vigorously for 10 seconds.

5. Incubate at 37° C. on a shaker for 30 minutes, also manually shaking the flasks vigorously for 5-10 seconds every 15 min.

6. On completion of the digestion period, the digested adipose tissue should have a “soup like” consistency.

7. Add patient's serum to each flask at a final concentration of 10% to stop collagenase activity.

### Phase 2:

### d.3—Separation of the Stromal-Vascular Fraction

After digestion, the ability of lipid-filled adipocytes to float is used to separate them from the stromal vascular fraction (SVF).

1. Dispense the collagenase-digested tissue into 50 ml tubes. Avoid dispensing undigested tissue.

Centrifuge at room temperature at 600×g for 10 min

2. After centrifugation, use a 50 ml pipette to aspirate the floating adipocytes, lipids and the digestion medium. Leave the SVF pellet in the tube.

3. Pool the pellets into two 50 ml tubes.

4. Suspend the pellet in 10 ml PBS.

5. Pipette the cells up and down several times to reduce clumping.

6. Allow undigested tissue clumps to settle by gravity for ˜1 min.

7. It is essential to obtain a cell suspension free from undigested tissue and cell clumps, so aspirate and pass the suspended cells through 100 μm cell strainers.

8. Pick 3 samples of 10 micro liter and count the number of cells based on hemocytometer method with trypan blue. Do not include RBCs in your counting.

9. Based on cell count, pick the appropriate volume of PBS+SVF solution containing at least 20 million cells up to maximum 50 million cells [103, 104].

10—Divide it equally in volume into two falcon tubes (15 ml).

11. Centrifuge the suspension at 600×g for 10 minute.

12. Remove the supernatant. Dissolve each plet in 0.2 ml sterile Ringer-lactate solution and drain it in a 2 ml sterile syringe with 18, ½ Gage needle.

### Phase 3

### d.4—Encapsulating the SVF in Fibrin Glue and Preparing for Injection:

Fibrin Glue is a two-component fibrin sealant that consists of human fibrinogen and human thrombin (Baxter Healthcare Corporation, Westlake Village, Calif.). When combined the two components mimic the final stage of the blood coagulation cascade. 2 sets of total 4 ml fibrin glue were picked and mixed each 0.2 ml SVF suspension with one of the 4 ml kit.

D.4.1. Preheat Fibrinotherm device

D.4.2. Take out the 4 vials.

D.4.3. Remove caps and place vials in Fibrinotherm device.

D.4.4. Disinfect visal stoppers and warm vials.

D.4.5. Spike the Thrombin vial, twist while spiking the Cacl₂ vial, pump plunger to drain vial. Remove transfer spike.

D.4.5. In the biosafety cabinet, Add the 0.2 ml SVF suspension to the 2 ml dissolved thrombin vial and shake it vigorously.

D.4.6. Bring the warmed the sealer protein concentrate vial and spike it.

D.4.7. Twist while spiking the Aprotinin vial.

D.4.8. Pump plunger to drain vial.

D.4.9. Remove transfer spike. Stir until dissolved

Then both components will be drained into two different 2 ml syringes and place them into the Duploject applicator. Since there were two sets of SVF and glue, a total volume of 8 ml including: two thrombin+SVF 2 ml syringes, and two fibrinogen (sealer protein) 2 ml syringes. Evaluate result.

e—Injection

Each of the two sets of thrombin+SVF suspension (2 ml) and fibrinogen (sealer protein) component (2 ml) will be injected over the PUL defect field at the same time by Duploject applicator.

### Example 3

### PUL Deficiency Causes SUI in Female Rats

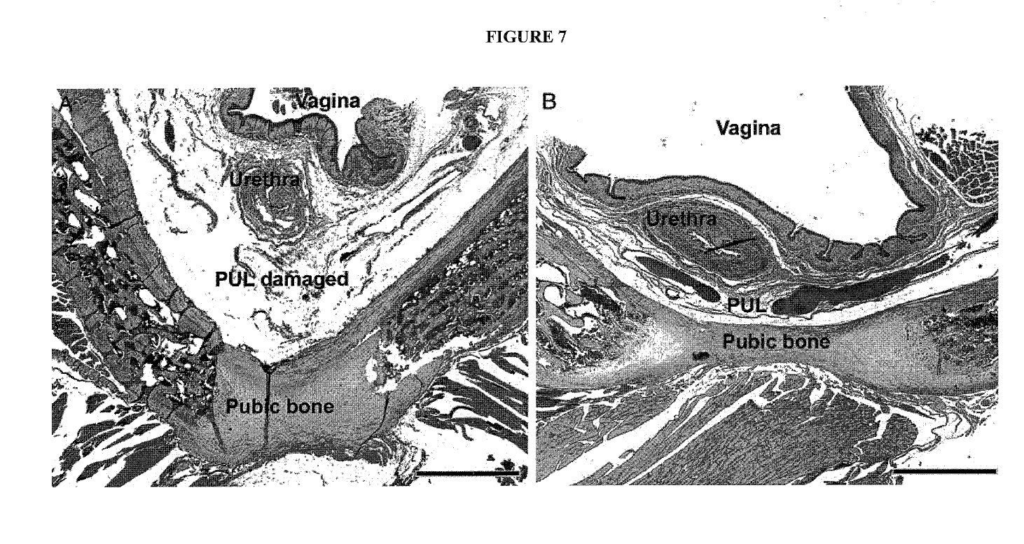

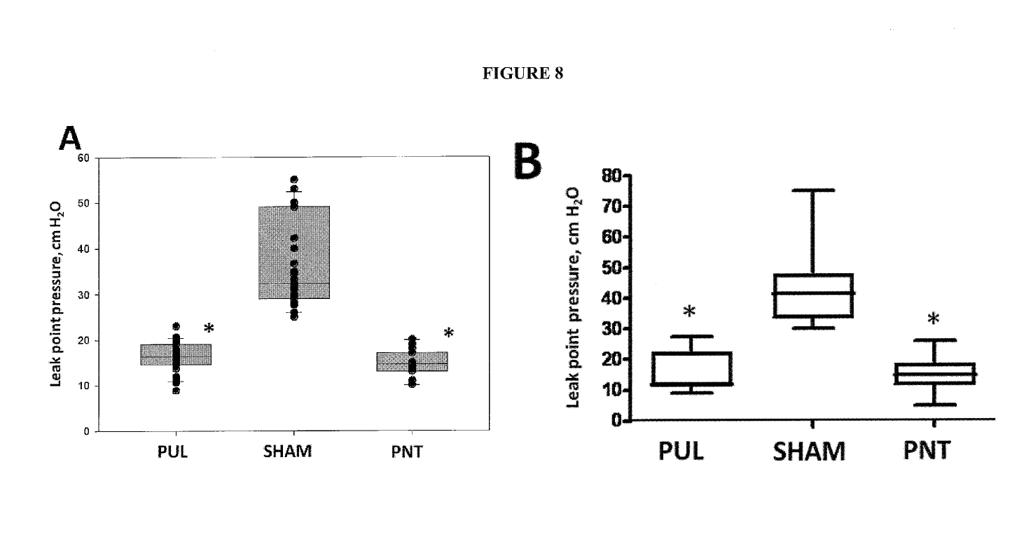

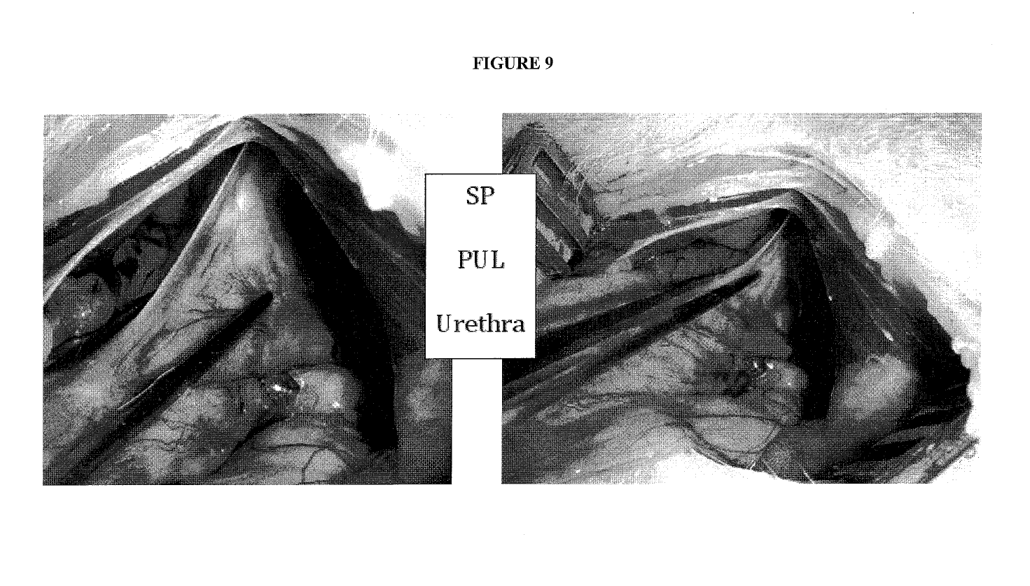

A model of SUI in female rats has been developed and described [56,57]. In summary, these published data confirm the phenotype and functional similarities of PUL between humans and female rats to the level that PUL injury causes SUI in rats (FIG. 7 and FIG. 8). In addition, the feasibility of injection of the material (Fibrin Glue and MSC) in Canine Models of PUL deficiency has been tested (FIG. 9).

### Example 4

### Efficacy and Feasibility of PUL Therapy

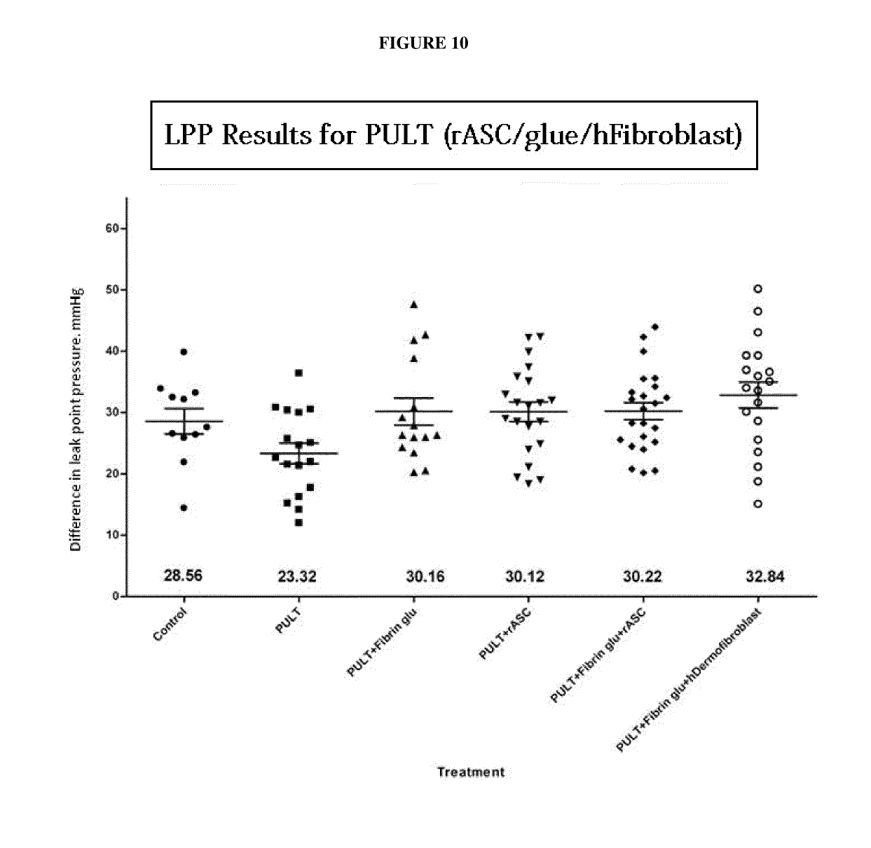

There are some studies to support the Efficacy and Feasibility of PUL Therapy. For instance, it was observed that rat adipose derived stem cells encapsulated in fibrin sealant could restore the continence in a rat model of SUI with PUL deficiency when injected in peri-urethra (FIG. 10).

### Example 5

### Restoring Continence in a Model of PUL Deficiency

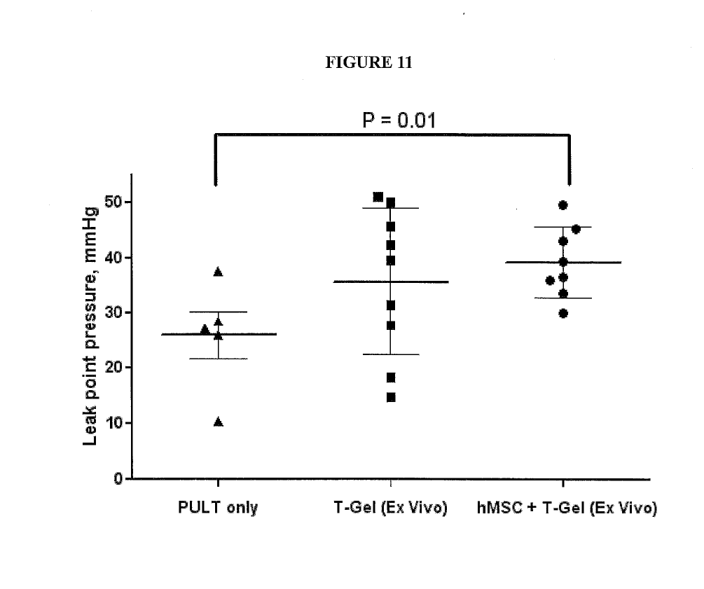

In another study, it was found that local administration of bone marrow derived human mesenchymal stem cells (hMSCs) and a dityramine cross-linked porcine collagen-based hydrogel (T-gel) could restore continence in a model of PUL deficiency (FIG. 11).

### REFERENCES

1. Bump, R. C. and Norton, P. A. (1998) “Epidemiology and natural history of pelvic floor dysfunction,” Obstet. Gynecol. Clin. North Am. 25(4), 723-746.
2. Norton, P. and Brubaker, L. (2006) “Urinary incontinence in women,” Lancet 367(9504), 57-67.
3. Deng, D. Y et al. (2007) “Presentation and management of major complications of midurethral slings: Are complications under-reported?,” Neurourol. Urodyn. 26(1), 46-52.
4. Di Lullo, G. A. et al. (2002) “Mapping the Ligand-binding Sites and Disease-associated Mutations on the Most Abundant Protein in the Human, Type I Collagen,” J. Biol. Chem. 277(6), 4223-4231.
5. Bell, E. et al. (1979) “Production of a tissue-like structure by contraction of collagen lattices by human fibroblasts of different proliferative potential in vitro,” P.N.A.S. 76(3), 1274-1278.
6. Helary, C. et al. (2010) “Concentrated collagen hydrogels as dermal substitutes,” Biomaterials 31(3), 481-490.
7. Darr, A. and Calabro, A. (2009) “Synthesis and characterization of tyramine-based hyaluronan hydrogels,” J. Mater Sci.: Mater. Med. 20(1), 33-44.
8. Nardi, N. B. and Meirelles, L. d. S. (2006) “Mesenchymal Stem Cells: Isolation, In Vitro Expansion and Characterization,” in Stem Cells (Wobus, A. and Boheler, K., Eds.), pp 249-282, Springer Berlin Heidelberg.
9. Brighton, C. T. and Hunt, R. M. (1991) “Early histological and ultrastructural changes in medullary fracture callus,” J. Bone Joint Surg. 73(6), 832-847.
10. Brighton, C. T. and Hunt, R. M. (1997) “Early Histologic and Ultrastructural Changes in Microvessels of Periosteal Callus,” J. Orthop. Trauma 11(4), 244-253.
11. Haliloglu, B. et al. (2010) “The role of urethral hypermobility and intrinsic sphincteric deficiency on the outcome of transobturator tape procedure: a prospective study with 2-year follow-up,” International Urogynecology Journal 21(2), 173-178.
12. Crepin, G et al. (2006) “The female urogenital system and high level sports,” Bull. Acad. Natl. Med. 190(7), 1479-1491.
13. Hood, A. F. et al. (1987) “Acute Graft-vs-Host Disease: Development Following Autologous and Syngeneic Bone Marrow Transplantation,” Arch. Dermatol. 123(6), 745-750.
14. Zittoun, R. A. et al. (1995) “Autologous or Allogeneic Bone Marrow Transplantation Compared with Intensive Chemotherapy in Acute Myelogenous Leukemia,” N. Engl. J. Med. 332(4), 217-223.
15. Yamada, Y. et al. (2003) “Bone regeneration following injection of mesenchymal stem cells and fibrin glue with a biodegradable scaffold,” Journal of Cranio-Maxillofacial Surgery 31(1), 27-33.
16. Ahmed, T. A. E. et al. (2011) “Fibrin glues in combination with mesenchymal stem cells to develop a tissue-engineered cartilage substitute,” Tissue Engineering, Part A: Tissue Engineering 17(3-4), 323-335.
17. Fang, H. et al. (2004) “Biocompatibility studies on fibrin glue cultured with bone marrow mesenchymal stem cells in vitro,” J. Huazhong Univ. Sci. Technolog. Med. Sci. 24(3), 272-274.
18. Roche, R. et al. (2010) “Stem cells for stress urinary incontinence: the adipose promise,” J. Cell. Mol. Med. 14(1-2), 135-142.
19. Furuta, A. et al. (2007) “Advances in the understanding of stress urinary incontinence and the promise of stem-cell therapy,” Rev. Urol. 9(3), 106-112.
20. Cheng, H. et al. “Fibrin Glue Composition for Repairing Nerve Damage and Methods Thereof,” United States Patent Application Publication Number US 2009-0191165 A1, application Ser. No. 12/354,470, filed Jan. 15, 2009. (published Jul. 30, 2009).
21. Motlagh, D. et al. “Using of Scaffold Comprising Fibrin for Delivery of Stem Cells,” United States Patent Application Publication Number US 2010-0028311 A1, application Ser. No. 12/500,582, filed Jul. 9, 2009. (published Feb. 4, 2010).
22. Milano, G. et al. “Method of Arthroscopic Osteochondral Resurfacing Using PRP Strengthened with Fibrin Glue,” United States Patent Application Publication Number US 2009-0062870 A1, application Ser. No. 12/198,836, filed Aug. 26, 2008. (published Mar. 5, 2009).
23. Chancellor, M. B. et al. “Rapid preparation of stem cell matrices for use in tissue and organ treatment and repair,” U.S. Pat. No. 7,906,110, application Ser. No. 11/138,168, filed May 25, 2005. (issued Mar. 15, 2011).
24. Schuldt, A. J. T. et al. “Fibroblast Derived Stem Cells,” United States Patent Application Publication Number US 2011-0041857 A1, application Ser. No. 12/744,568, filed Nov. 26, 2008. (published Feb. 24, 2011).
25. Fu, Y-s. “Treatment of spinal injuries using human umbilical mesenchymal stem cells,” United States Patent Application Publication Number US 2008-0305148 A1, application Ser. No. 12/047,417, filed Mar. 13, 2008. (published Dec. 11, 2008).
26. Lendeckel, S. et al. (2004) “Autologous stem cells (adipose) and fibrin glue used to treat widespread traumatic calvarial defects: case report,” J. Craniomaxillofac. Surg. 32(6), 370-373.
27. Daneshgari, F. (2009) “Re: FDA Public Health Notification: Serious Complications Associated with Transvaginal Placement of Surgical Mesh in Repair of Pelvic Organ Prolapse and Stress Urinary Incontinence,” Eur Urol. 55(5), 1235-1236.
28. Daneshgari, F. and Moore, C. (2006) “Advancing the understanding of pathophysiological rationale for the treatment of stress urinary incontinence in women: the ‘trampoline theory’,” BJU Int. 98, 8-14.
29. Daneshgari, F. et al. (2008) “Complications of Mid Urethral Slings: Important Outcomes for Future Clinical Trials,” J. Urol. 180(5), 1890-1897.
30. Carr, L. et al. (2008) “1-year follow-up of autologous muscle-derived stem cell injection pilot study to treat stress urinary incontinence,” International Urogynecology Journal 19(6), 881-883.
31. Strasser, H. et al. (2007) “Transurethral ultrasonography-guided injection of adult autologous stem cells versus transurethral endoscopic injection of collagen in treatment of urinary incontinence,” World J. Urol. 25(4), 385-392.
32. Smaldone, M. and Chancellor, M. (2008) “Muscle derived stem cell therapy for stress urinary incontinence,” World J. Urol. 26(4), 327-332.
33. Fu, Q. et al. (2010) “Myoblasts differentiated from adipose-derived stem cells to treat stress urinary incontinence,” Urology 75(3), 718-723.
34. Guiting, L. et al. (2010) “Treatment of stress urinary incontinence with adipose tissue-derived stem cells,” Cytotherapy 12(1), 88-95.
35. Yamamoto, T. et al. (2010) “Periurethral injection of autologous adipose-derived stem cells for the treatment of stress urinary incontinence in patients undergoing radical prostatectomy: Report of two initial cases,” Int. J. Urol. 17(1), 75-82.
36. Zhao, W. et al. (2011) “Periurethral Injection of Autologous Adipose-Derived Stem Cells with Controlled-Release Nerve Growth Factor for the Treatment of Stress Urinary Incontinence in a Rat Model,” Eur. Urol. 59(1), 155-163.
37. Kim, S. O. et al. (2011) “Bone-marrow-derived mesenchymal stem cell transplantation enhances closing pressure and leak point pressure in a female urinary incontinence rat model,” Urol. Int. 86(1), 110-116.
38. Corcos, J. et al. (2011) “Bone marrow mesenchymal stromal cell therapy for external urethral sphincter restoration in a rat model of stress urinary incontinence,” Neurourol. Urodyn. 30(3), 447-455.
39. Zou, X. H. et al. (2010) “Mesenchymal stem cell seeded knitted silk sling for the treatment of stress urinary incontinence,” Biomaterials 31(18), 4872-4879.
40. Casteilla, L. et al. (2011) “Adipose-derived stromal cells: Their identity and uses in clinical trials, an update,” World Journal of Stem Cells 3(4), 25-33.
41. Black, L. et al. (2007) “Effect of adipose-derived mesenchymal stem and regenerative cells on lameness in dogs with chronic osteoarthritis of the coxofemoral joints: a randomized, double-blinded, multicenter, controlled trial.,” Vet. Ther. 8(4), 272-284.
42. Black, L. et al. (2008) “Effect of intraarticular injection of autologous adipose-derived mesenchymal stem and regenerative cells on clinical signs of chronic osteoarthritis of the elbow joint in dogs,” Vet. Ther 9(3), 192-200.
43. Guisantes, E. et al. (2011) “Autologous Fat Grafting for Unaesthetic Scars Correction,” Ann. Plast. Surg. 67, May 27 epub.
44. Zuk, P. A. et al. (2001) “Multilineage Cells from Human Adipose Tissue: Implications for Cell-Based Therapies,” Tissue Eng. 7(2), 211-228.
45. Zuk, P. A. et al. (2002) “Human Adipose Tissue Is a Source of Multipotent Stem Cells,” Mol. Biol. Cell 13(12), 4279-4295.
46. Astori, G et al. (2010) “Bone marrow derived stem cells in regenerative medicine as Advanced Therapy Medicinal Products,” American Journal of Translational Research 2(3), 285-295.
47. Odegaard, J. I. et al. (2007) “Macrophage-specific PPAR[ggr] controls alternative activation and improves insulin resistance,” Nature 447(7148), 1116-1120.
48. Brown, J. S. et al. (2006) “Prevalence and risk factors for urinary incontinence in women with type 2 diabetes and impaired fasting glucose: findings from the National Health and Nutrition Examination Survey (NHANES) 2001-2002,” Diabetes Care 29(6), 1307-1312.
49. Daneshgari, F. (2009) “Words of wisdom. Re: FDA public health notification: serious complications associated with transvaginal placement of surgical mesh in repair of pelvic organ prolapse and stress urinary incontinence,” Eur. Urol. 55(5), 1235-1236.
50. Peyrat, L. et al. (2002) “Prevalence and risk factors of urinary incontinence in young and middle-aged women,” BJU Int. 89(1), 61-66.
51. Hampel, C. et al. (1997) “Prevalence and natural history of female incontinence,” Eur Urol. 32 Suppl 2, 3-12.
52. Petros, P. E. and Woodman, P. J. (2008) “The Integral Theory of continence,” Int. Urogynecol. J. Pelvic Floor Dysfunct. 19(1), 35-40.
53. Petros, J. G and Bradley, T. M. (1990) “Factors influencing postoperative urinary retention in patients undergoing surgery for benign anorectal disease,” Am. J. Surg. 159(4), 374-376.
54. Petros, P. E. and Ulmsten, U. I. (1990) “An integral theory of female urinary incontinence. Experimental and clinical considerations,” Acta Obstetrica et Gynaecologica Scandinavica. Supplement 153, 7-31.
55. Petros, P. P. and Ulmsten, U. (1998) “An anatomical classification—a new paradigm for management of female lower urinary tract dysfunction,” Eur. J. Obstet. Gynecol. Reprod. Biol. 80(1), 87-94.
56. Kefer, J. C. et al. (2008) “Pubo-Urethral Ligament Transection Causes Stress Urinary Incontinence in the Female Rat: A Novel Animal Model of Stress Urinary Incontinence,” J. Urol. 179(2), 775-778.
57. Kefer, J. C. et al. (2009) “Pubo-Urethral Ligament Injury Causes Long-Term Stress Urinary Incontinence in Female Rats: An Animal Model of the Integral Theory,” J. Urol. 181(1), 397-400.
58. Hijaz, A. et al. (2004) “Efficacy of a vaginal sling procedure in a rat model of stress urinary incontinence,” J. Urol. 172(5, Part 1), 2065-2068.
59. Chen, C. C. G et al. (2009) “Collagen remodeling and suburethral inflammation might account for preserved anti-incontinence effects of cut polypropylene sling in rat model,” Urology 73(2), 415-420.
60. Hijaz, A. et al. (2005) “Role of sling integrity in the restoration of leak point pressure in the rat vaginal sling model,” J. Urol. 174(2), 771-775.
61. Daneshgari, F. (2011) “Editorial Comment,” Urology 78(2), 299-300.
62. Groen, J. and Bosch, J. L. (2004) “Bladder contraction strength parameters poorly predict the necessity of long-term catheterization after a pubovaginal rectus fascial sling procedure,” J. Urol. 172(3), 1006-1009.
63. DeLancey, J. O. et al. (2008) “Stress urinary incontinence: relative importance of urethral support and urethral closure pressure,” J. Urol. 179(6), 2286-2290; discussion 2290.
64. Furuta, A. et al. (2007) “State of the art of where we are at using stem cells for stress urinary incontinence,” Neurourol. Urodyn. 26(7), 966-971.
65. Quevedo, H. C. et al. (2009) “Allogeneic mesenchymal stem cells restore cardiac function in chronic ischemic cardiomyopathy via trilineage differentiating capacity,” P.N.A.S. 106(33), 14022-14027.
66. Tettamanti, G et al. (2004) “The multifunctional role of fibroblasts during wound healing in Hirudo medicinalis (Annelida, Hirudinea),” Biol. Cell 96(6), 443-455.
67. Cool, S. M. et al. (2004) “Temporal expression of fibroblast growth factor receptors during primary ligament repair,” Knee Surg. Sports Traumatol. Arthrosc. 12(5), 490-496.
68. Yiou, R. et al. (2002) “Muscle precursor cell autografting in a murine model of urethral sphincter injury,” BJU Int. 89(3), 298-302.
69. Wu, G et al. (2011) “Adipose-derived stromal cell transplantation for treatment of stress urinary incontinence,” Tissue Cell 43(4), 246-253.
70. Masuda, S. et al. (2008) “Cell sheet engineering for heart tissue repair,” Adv. Drug Delivery Rev. 60(2), 277-285.
71. Zhang, X. et al. (2010) “Preservation of the cardiac function in infarcted rat hearts by the transplantation of adipose-derived stem cells with injectable fibrin scaffolds,” Exp. Biol. Med. 235(12), 1505-1515.
72. Baumgartner, L. et al. (2010) “Human mesenchymal stem cells: Influence of oxygen pressure on proliferation and chondrogenic differentiation in fibrin glue in vitro,” J. Biomed. Mater. Res. A 93(3), 930-940.
73. Christman, K. L. et al. (2003) “Delivery of pleiotrophin plasmid induces neovasculature formation in ischemic myocardium,” Mol. Ther. 7(5), 5231-S231.
74. Itosaka, H. et al. (2009) “Fibrin matrix provides a suitable scaffold for bone marrow stromal cells transplanted into injured spinal cord: a novel material for CNS tissue engineering,” Neuropathology 29(3), 248-257.
75. Spotnitz, W. D. (2010) “Fibrin sealant: past, present, and future: a brief review,” World J. Surg. 34(4), 632-634.
76. Chen, Y. T. et al. (2011) “Adipose-derived mesenchymal stem cell protects kidneys against ischemia-reperfusion injury through suppressing oxidative stress and inflammatory reaction,” J. Transl. Med. 9, 51.
77. Shamliyan, T. A. et al. (2008) “Systematic review: randomized, controlled trials of nonsurgical treatments for urinary incontinence in women,” Ann. Intern. Med. 148(6), 459-473.
78. Ken, L. A. (2005) “Bulking agents in the treatment of stress urinary incontinence: history, outcomes, patient populations, and reimbursement profile,” Rev. Urol. 7 Suppl 1, S3-S11.
79. Gilchrist, A. S. and Rovner, E. S. (2012) “Sling location in women with recurrent stress urinary incontinence following midurethral sling,” Urology 79(1), 76-79.
80. Chancellor, M. B. et al. (2000) “Preliminary results of myoblast injection into the urethra and bladder wall: a possible method for the treatment of stress urinary incontinence and impaired detrusor contractility,” Neurourol. Urodyn. 19(3), 279-287.
81. Lin, C. S. and Lue, T. F. (2012) “Stem cell therapy for stress urinary incontinence: a critical review,” Stem Cells Dev. 21(6), 834-843.
82. Lee, C. N. et al. (2010) “Human cord blood stem cell therapy for treatment of stress urinary incontinence,” J. Korean Med. Sci. 25(6), 813-816.
83. De Ugarte, D. A. et al. (2003) “Comparison of multi-lineage cells from human adipose tissue and bone marrow,” Cells Tissues Organs 174(3), 101-109.
84. Little, D. et al. (2010) “Ligament-derived matrix stimulates a ligamentous phenotype in human adipose-derived stem cells,” Tissue Eng. Part A 16(7), 2307-2319.
85. Lin, G et al. (2010) “Treatment of stress urinary incontinence with adipose tissue-derived stem cells,” Cytotherapy 12(1), 88-95.
86. Colazzo, F. et al. (2011) “Extracellular matrix production by adipose-derived stem cells: implications for heart valve tissue engineering,” Biomaterials 32(1), 119-127.
87. Sandor, G. K. and Suuronen, R. (2008) “Combining adipose-derived stem cells, resorbable scaffolds and growth factors: an overview of tissue engineering,” J. Can. Dent. Assoc. 74(2), 167-170.
88. Yang, J. Q. et al. (2005) “[Effect of shenqi fuzheng injection on pre/post-operational change of argyrophilic-nucleolar organizer regions in peripheral T-lymphocyte in patients with gastric carcinoma],” Zhongguo Zhong Xi Yi Jie He Za Zhi 25(7), 626-628.
89. Spotnitz, W. D. and Burks, S. (2008) “Hemostats, sealants, and adhesives: components of the surgical toolbox,” Transfusion (Paris) 48(7), 1502-1516.
90. Garcia-Olmo, D. et al. (2009) “Expanded adipose-derived stem cells for the treatment of complex perianal fistula: a phase II clinical trial,” Dis. Colon Rectum 52(1), 79-86.
91. Kretlow, J. D. et al. (2010) “Uncultured marrow mononuclear cells delivered within fibrin glue hydrogels to porous scaffolds enhance bone regeneration within critical-sized rat cranial defects,” Tissue Eng. Part A 16(12), 3555-3568.
92. Farhat, W. A. et al. (2006) “Impact of fibrin glue and urinary bladder cell spraying on the in-vivo acellular matrix cellularization: a porcine pilot study,” Can. J. Urol. 13(2), 3000-3008.
93. Becker, J. C. et al. (2004) “Biological in vitro effects of fibrin glue: fibroblast proliferation, expression and binding of growth factors,” Scand. J. Gastroenterol. 39(10), 927-932.
94. Aoyagi, Y. et al. (2011) “Fibrin glue increases the cell survival and the transduced gene product secretion of the ceiling culture-derived adipocytes transplanted in mice,” Exp. Biol. Med. 43(3), 161-167.
95. Nair, M. B. et al. (2009) “Platelet-rich plasma and fibrin glue-coated bioactive ceramics enhance growth and differentiation of goat bone marrow-derived stem cells,” Tissue Eng. Part A 15(7), 1619-1631.
96. Alston, S. M. et al. (2007) “New method to prepare autologous fibrin glue on demand,” Transl. Res. 149(4), 187-195.
97. Bernardo, M. E. et al. (2012) “Mesenchymal stromal cell therapy: a revolution in Regenerative Medicine?,” Bone Marrow Transplant. 47(2), 164-171.
98. Dai, W. et al. (2005) “Allogeneic Mesenchymal Stem Cell Transplantation in Postinfarcted Rat Myocardium,” Circulation 112(2), 214-223.
99. Gimble, J. et al. (2011) “Isolation and growth of stem cells,” in Tissue Engineering: From Lab to Clinic (Pallua, N. and Suscheck, C., Eds.), pp 93-111, Springer-Verlag, Berlin.
100. McGuire, E. and English, S. (1997) “Periurethral collagen injection for male and female sphincteric incontinence: indications, techniques, and result,” World J. Urol. 15(5), 306-309.
101. Garcí a-Olmo, D. et al. (2005) “A Phase I Clinical Trial of the Treatment of Crohn's Fistula by Adipose Mesenchymal Stem Cell Transplantation,” Dis. Colon Rectum 48(7), 1416-1423.
102. De Ugarte, D. A. et al. (2003) “Differential expression of stem cell mobilization-associated molecules on multi-lineage cells from adipose tissue and bone marrow,” Immunol. Lett 89(2-3), 267-270.
103. Ichim, T. E. et al. (2010) “Autologous stromal vascular fraction cells: A tool for facilitating tolerance in rheumatic disease,” Cell. Immunol. 264(1), 7-17.
104. Riordan, N. et al. (2009) “Non-expanded adipose stromal vascular fraction cell therapy for multiple sclerosis,” J. Transl. Med. 7(1), 1-9.
105. Cytori Therapeutics. (2007) “A Randomized Clinical Trial of AdiPOse-derived Stem ceLLs in the Treatment of Patients With ST-elevation myOcardial Infarction—The APOLLO Trial,” p ClinicalTrials.gov Identifier: NCT00442806.
106. Cytori Therapeutics. (2007) “A Randomized Clinical Trial of adiPose-deRived stEm & Regenerative Cells In the Treatment of Patients With Non revaScularizable ischEmic Myocardium—The PRECISE Trial,” p ClinicalTrials.gov Identifier: NCT00426868.
107. Adistem Ltd. (2008) “Phase I/II Study of Intravenous Administration of Activated Autologous Adipose-Derived Stromal Vascular Fraction in Patients With Type 1 Diabetes,” p ClinicalTrials.gov Identifier: NCT00703599.
108. Kanazawa University and Cytori Therapeutics. (2009) “Liver Regeneration Therapy Using Autologous Adipose Tissue Derived Stromal Cells,” p ClinicalTrials.gov Identifier: NCT00913289.
109. Williams, S. K. et al. (1995) “Collagenase lot selection and purification for adipose tissue digestion,” Cell Transplant. 4(3), 281-289.

We claim:

1. A method, comprising:
a) providing:
i) a subject exhibiting at least one symptom of urinary incontinence due to pubo-urethral ligament damage;
ii) a composition comprising at least one fibrin glue and an amount of meschencymal stem cells;
b) contacting said composition with said pubo-urethral ligament damage; and
c) reducing said at least one symptom of urinary incontinence.

2. The method according to claim 1, wherein said fibrin glue comprises elements selected from the group consisting of fibrinogen, collagen, collagen hydrogels, and tyramine substituted enzymatically cross-linkable collagen gels.

3. The method according to claim 1, wherein said meschencymal stem cells are size-sieved stem cells (SSCs) with a diameter equal or greater than 3-μm.

4. The method according to claim 1, wherein said contacting is topical.

5. The method according to claim 1, wherein said reducing said at least one symptom enhances, at least partially, a functional recovery from urinary incontinence.

6. The method according to claim 1, wherein said reducing said at least one symptom improves urine retention.

7. The method according to claim 1, wherein said reducing said at least one symptom improves urine release.

8. The method according to claim 1, wherein said pubo-urethral ligament damage is caused by childbirth trauma.

9. The method according to claim 1, wherein said pubo-urethral ligament damage is caused by prostate surgery.

10. The method according to claim 1, wherein said meschencymal stem cells are derived from bone marrow or adipose tissue.

11. The method according to claim 1, wherein said urinary incontinence is stress urinary incontinence.

12. The method according to claim 1, wherein said fibrin glue composition is fresh.

13. A composition comprising a fibrin glue and an amount of meschencymal stem cells.

14. The composition of claim 13, wherein said fibrin glue comprises at least one element selected from the group consisting of fibrinogen, collagen, collagen hydrogels, and tyramine substituted enzymatically cross-linkable collagen gels.

15. The composition of claim 13, wherein said MSCs are size-sieved stem cells with a diameter equal or greater than 3-μm.
